(19) United States
(12) Patent Application Publication
(10) Pub. No.: US 2026/0041394 A1
(43) Pub. Date: Feb. 12, 2026

(54) Nonlinear Contrast Imaging with Ultrasound

(71) Applicant: FUJIFILM SonoSite, Inc., Bothell, WA (US)

(72) Inventors: John Ross Williams, Toronto (CA); Alex Forbrich, Toronto (CA); Lauren Wirtzfeld, Thornhill (CA); William Andrew Needles, Toronto (CA)

(73) Assignee: FUJIFILM SonoSite, Inc., Bothell, WA (US)

(21) Appl. No.: 18/797,865

(22) Filed: Aug. 8, 2024

(51) Int. Cl. A61B 8/06; A61B 8/00; A61B 8/08

(52) U.S. Cl. CPC A61B 8/06; A61B 8/469; A61B 8/481

(57) ABSTRACT

Systems and methods for nonlinear contrast imaging with ultrasound are described, including an ultrasound device that generates a transmission signal, which includes a first ultrasound pulse and a second ultrasound pulse. The first pulse and the second pulse overlap along a bisector at a region of interest (ROI). The second pulse is a phase-inverted version of the first pulse. A reception signal is generated with linear and nonlinear components (e.g., nonlinear reflections from microbubbles). The linear components of the reception signal cancel upon combination (e.g., summation), leaving the nonlinear components for imaging. Multi-line imaging is also possible using one or more additional pulse pairs. The use of two full-amplitude, phase-inverted pulses enables improved signal-to-noise ratio (SNR), depth penetration, and increased frame rate, while minimizing resource utilization over a three-pulse amplitude-modulation-based approach. The ability to use a multi-line acquisition scheme further enhances these advantages.

### BACKGROUND

Ultrasound imaging of blood flow using a contrast agent (e.g., microbubbles) can be confounded by tissue harmonics. At high ultrasound frequencies (e.g., greater than 20 MHz), the tissue harmonics can be particularly problematic due to the increased pressures that result when trying to overcome attenuation losses. Some approaches to solving the problem of tissue harmonics in contrast imaging with ultrasound involve plane-wave imaging. For example, a hybrid of plane-wave imaging and single-line beamforming can attempt to reduce tissue harmonics with three separate transmit events. The approach uses a three-pulse, amplitude-modulation (AM) scheme with cross-propagating beams to reduce the pressure levels outside the region of overlap, thereby reducing the generation of tissue harmonic signals and consequently improving the contrast-to-tissue ratio.

Since this approach utilizes plane-wave imaging, the penetration can be poor. Further, because the AM scheme uses half-amplitude pulses, the signal-to-noise ratio (SNR) is low. In addition, because this approach uses three separate transmit events, the frame rate is reduced and resource requirements are increased. A robust solution is needed to realize higher SNR, avoid frame rate reduction, and have good penetration.

### SUMMARY

Systems and methods for nonlinear contrast imaging with ultrasound are described, including an ultrasound device that generates a transmission signal, which includes a first ultrasound pulse and a second ultrasound pulse. The first pulse and the second pulse overlap along a bisector at a region of interest (ROI). The second pulse is a phase-inverted version of the first pulse. A reception signal is generated with linear and nonlinear components (e.g., nonlinear reflections from microbubbles). The linear components of the reception signal cancel upon combination (e.g., summation), leaving the nonlinear components for imaging. Multi-line imaging is also possible using one or more additional pulse pairs. The use of two full-amplitude, phase-inverted pulses enables improved signal-to-noise ratio (SNR), depth penetration, and increased frame rate, while minimizing resource utilization over a three-pulse amplitude-modulation-based approach. The ability to use a multi-line acquisition scheme further enhances these advantages

In some implementations, an ultrasound device is disclosed. The ultrasound device includes an ultrasound scanner, the ultrasound scanner configured to generate a first ultrasound signal with a first polarity and a second ultrasound signal with a second polarity, the second polarity being an inverse of the first polarity. The ultrasound scanner is further configured to transmit the first ultrasound signal and the second ultrasound signal at an ROI of a subject, receive a first return signal based on the first ultrasound signal reflecting from the ROI, and receive a second return signal based on the second ultrasound signal reflecting from the ROI. The ultrasound device further includes one or more processors and a memory. The memory stores instructions that, when executed by the one or more processors, cause the one or more processors to generate a combined signal based on the first return signal and the second return signal and generate an output based on the combined signal.

According to some examples, the ultrasound scanner is further configured to generate a third ultrasound signal with the first polarity and a fourth ultrasound signal with the second polarity. In some examples, the ultrasound scanner is further configured to transmit the third ultrasound signal and the fourth ultrasound signal at the ROI, receive a third return signal based on the third ultrasound signal reflecting from the ROI, and receive a fourth return signal based on the fourth ultrasound signal reflecting from the ROI. In some examples, the instructions further cause the one or more processors to generate a second combined signal based on the third return signal and the second return signal and a second output based on the second combined signal.

In some implementations, a method for nonlinear contrast imaging with ultrasound is described. The method includes generating, by an ultrasound scanner, a first ultrasound signal with a first polarity and a second ultrasound signal with a second polarity. In aspects, the second polarity is an inverse of the first polarity. The method further includes transmitting, by the ultrasound scanner, the first ultrasound signal and the second ultrasound signal at an ROI of a subject. The method further includes receiving, by the ultrasound scanner, a first return signal based on the first ultrasound signal reflecting from the ROI, and a second return signal based on the second ultrasound signal reflecting from the ROT. The method further includes generating, by one or more processors, a combined signal based on the first return signal and the second return signal and an output based on the combined signal.

In some examples, the first ultrasound signal includes a first pulse and the second ultrasound signal includes a second pulse. The generation of the combined signal, in some examples, represents a beamforming of the first pulse and the second pulse at the ROT. According to some examples, the method further includes changing, by the one or more processors, one or more parameters of the first ultrasound signal or the second ultrasound signal. The changing of the one or more parameters, in some examples, is based on at least one of the first return signal or the second return signal. The one or more parameters include a phase of the first pulse or the second pulse, an amplitude of the first pulse or the second pulse, a frequency of the first pulse or the second pulse, or a waveform of the first pulse or the second pulse. The changing of the one or more parameters, according to some examples, is responsive to a user input. In some examples, the changing of the one or more parameters is based on a machine-learned model.

In aspects, the first return signal includes a first linear component and a first nonlinear component and the second return signal includes a second linear component and a second nonlinear component. The generating of the combined signal, in some examples, is based on a linear combination of the first return signal and the second return signal, and the first linear component is substantially opposite to the second linear component. In aspects, the transmitting of the first ultrasound signal and the second ultrasound signal at the ROI includes determining, by the ultrasound scanner, a coverage area of the transmission of the first ultrasound signal and the second ultrasound signal and a bisector of the coverage area. In some examples, the first ultrasound signal and the second ultrasound signal are configured to converge at a point on the bisector.

In some implementations, a user interface for an ultrasound device is disclosed. The user interface is configured to cause an ultrasound scanner of the ultrasound device to generate a first ultrasound signal with a first polarity and a second ultrasound signal with a second polarity. In aspects, the second polarity is an inverse of the first polarity, and the user interface is further configured to transmit the first ultrasound signal and the second ultrasound signal at a region of interest (ROI) of a subject, receive a first return signal based on the first ultrasound signal reflecting from the ROI, and receive a second return signal based on the second ultrasound signal reflecting from the ROI. The user interface is further configured to cause one or more processors of the ultrasound device to generate a combined signal based on the first return signal and the second return signal and an output based on the combined signal.

Other devices and methods to provide nonlinear contrast imaging with ultrasound are also described. These other devices and methods, in addition to those already disclosed, can be combined to generate additional devices and methods, which, though not explicitly disclosed herein, still are the same in concept as the devices and methods described explicitly herein. The devices and methods explicitly outlined herein are meant to be illustrative and not limiting.

### BRIEF DESCRIPTION OF THE DRAWINGS

The appended drawings illustrate examples and are, therefore, exemplary embodiments and not considered to be limiting in scope.

FIG. 1 illustrates an example environment for an ultrasound machine for nonlinear contrast imaging with ultrasound.

FIG. 2A illustrates an example implementation of the ultrasound machine from FIG. 1.

FIG. 2B illustrates another example implementation of the ultrasound machine from FIG. 1.

FIG. 3 illustrates an example ultrasound transducer for nonlinear contrast imaging with ultrasound.

FIG. 4 illustrates an example of waveform combinations for nonlinear contrast imaging with ultrasound.

FIG. 5 illustrates example comparison results using nonlinear contrast imaging with ultrasound.

FIG. 6 illustrates an example user interface for nonlinear contrast imaging with ultrasound.

FIG. 7 illustrates an example contrast imaging control panel from the user interface of FIG. 6.

FIG. 8 illustrates an example ultrasound transducer for nonlinear contrast imaging with ultrasound using multi-line scanning.

FIG. 9A illustrates an outline of a multi-line embodiment for nonlinear contrast imaging with ultrasound.

FIG. 9B illustrates an outline of a multi-line embodiment without a central line for nonlinear contrast imaging with ultrasound.

FIG. 10 outlines a method for nonlinear contrast imaging with ultrasound.

FIG. 11 outlines a method for multi-line nonlinear contrast imaging with ultrasound.

### DETAILED DESCRIPTION

Ultrasound imaging of blood flow using a contrast agent (e.g., microbubbles) can be confounded by tissue harmonics. At high ultrasound frequencies (e.g., greater than 20 MHz), the tissue harmonics can be particularly problematic due to the increased pressures that result when trying to overcome attenuation losses. Hence, a patient may not receive the best care possible. Accordingly, systems, devices, and methods are disclosed herein that increase penetration and signal to noise ratio (SNR) when imaging with the contrast agent without sacrificing frame rate, resulting in a clearer and more accurate ultrasound process compared to other ultrasound systems.

Moreover, an increase in resolution, frame rate, and/or contrast of a target for imaging enables a reduction in undesirable effects. Undesirable effects can include imaging of elements other than the target for imaging, low resolution, artifacts in the imaging, or any number of other unwanted or unideal outcomes from an ultrasound examination. By reducing the number and/or severity of undesirable effects, nonlinear contrast imaging with ultrasound enhances the efficacy of an ultrasound examination and related diagnoses.

### Example System

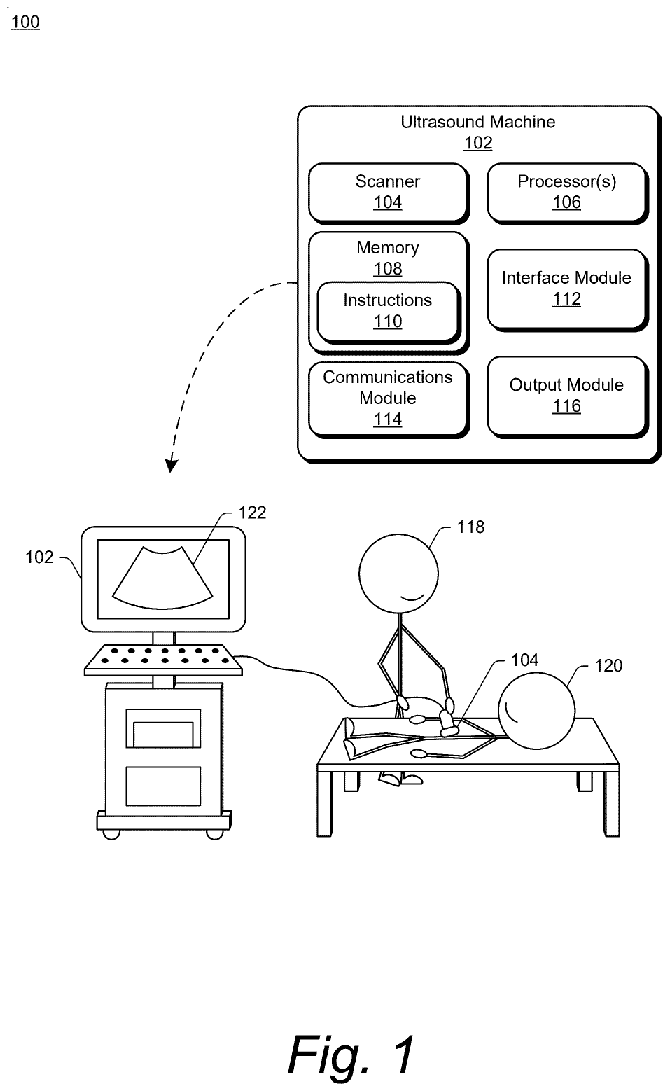

FIG. 1 illustrates an example environment 100 for an ultrasound machine 102 for nonlinear contrast imaging with ultrasound, in accordance with one or more implementations. Generally, the ultrasound machine 102 includes various components, some of which include a scanner 104, one or more processors 106, a memory 108 storing instructions 110, an interface module 112, a communications module 114, and an output module 116. Other components not pictured may also be included. In some examples, the interface module 112 is a user input device (e.g., a keyboard, a cursor control device, a microphone, a camera, etc.). In some examples, the communications module 114 includes one or more wireless transmitters, receivers, or transceivers over a wireless connection or network (e.g., Bluetooth™, Wi-Fi™, etc.). In other examples, the communications module 114 includes a wired connection.

A user 118 (e.g., nurse, ultrasound technician, clinician, operator, sonographer, etc.) directs the scanner 104 toward a patient 120 to non-invasively scan internal bodily structures (e.g., anatomies, organs, tissues, etc.) of the patient 120 for testing, diagnostic, or therapeutic reasons. In some implementations, the scanner 104 includes an ultrasound transducer array and electronics communicatively coupled to the ultrasound transducer array to transmit ultrasound signals to the patient's anatomy and receive ultrasound signals reflected from the patient's anatomy (e.g., echoes). In some implementations, the scanner 104 is an ultrasound scanner, which can also be referred to as an ultrasound probe or transducer.

In aspects, the scanner 104 is configured to produce nonlinear contrast imaging with ultrasound, such as by generating a first ultrasound signal and a second ultrasound signal. The first ultrasound signal, in aspects, has a first polarity. For example, the first polarity can be a first waveform. The second ultrasound signal, in aspects, has a second polarity, which is opposite to the first polarity. In the example where the first polarity represents a waveform, the second polarity represents a waveform that is the negative of the waveform of the first polarity. This can be represented mathematically as:

$${ψ}_{1} = - {ψ}_{2} \qquad Eq . 1$$

The term ψ₁ in Eq. 1 is the first waveform and ψ₂ in Eq. 1 is the second waveform. It is clear to see that ψ₁+ψ₂=0. Eq. 1 can be satisfied by defining ψ₂ as the negative of ψ₁, as a phase-shifted ψ₁, as a phase-inverted ψ₁, or by any other means known to a person in the art. For example, if ψ₁=sin(x), a valid form for ψ₂ using a phase shift is ψ₂=sin(x+π).

In some examples, the output module 116 is a display such as a display 122. The output module 116 is coupled to the one or more processors 106, which process the reflected ultrasound signals to generate ultrasound data. The output module 116 is, according to some examples, configured to generate and display an ultrasound image of the anatomy based on the ultrasound data generated by the one or more processors 106 from the reflected ultrasound signals detected by the scanner 104. In aspects, the ultrasound data includes the ultrasound image or data representing the ultrasound image.

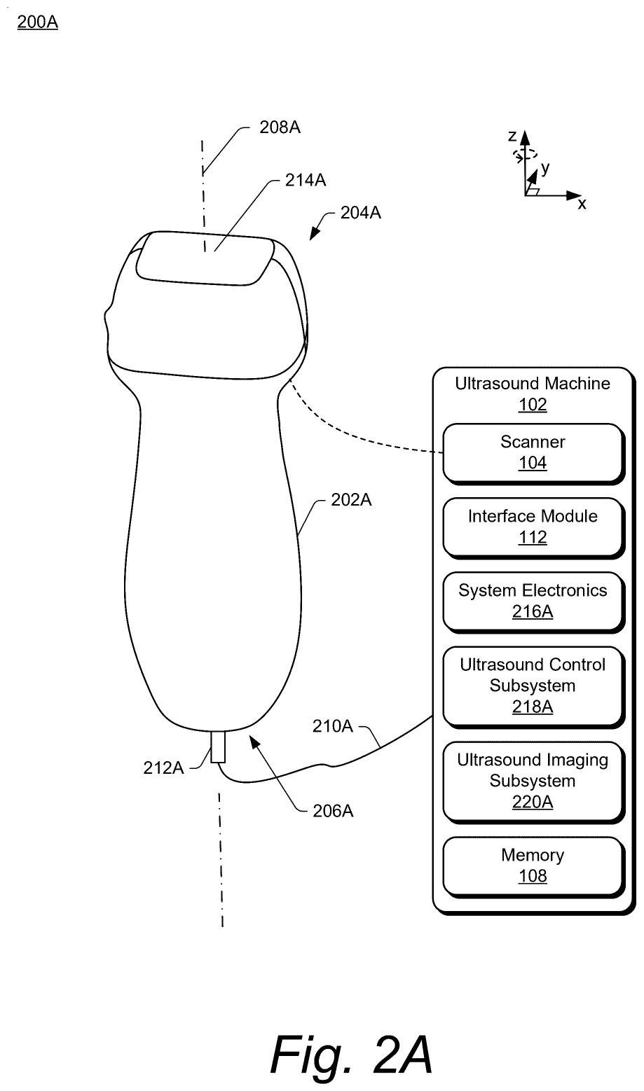

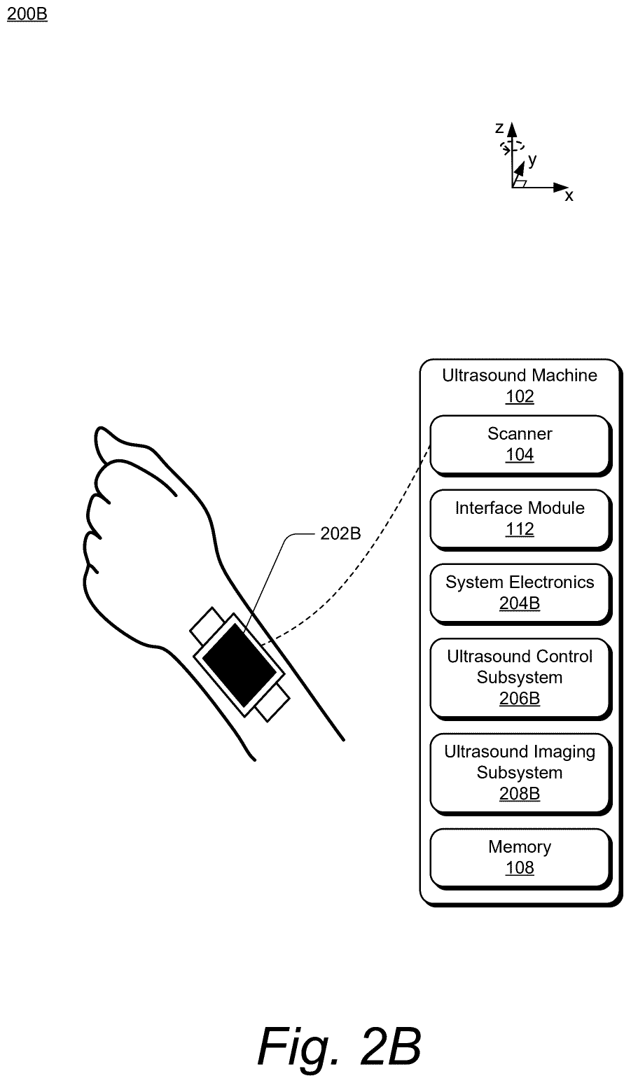

FIG. 2A illustrates an example implementation 200A of the ultrasound machine 102 from FIG. 1. The scanner 104 (e.g., ultrasound scanner) includes an enclosure 202A extending between a distal end portion 204A and a proximal end portion 206A. The enclosure 202A includes a central axis 208A (e.g., longitudinal axis) that intersects the distal end portion 204 and the proximal end portion 206A. The central axis 208A corresponds to an axial direction of the scanner 104. In an example, the scanner 104 is electrically coupled to an ultrasound imaging system (e.g., the ultrasound machine 102) via a cable 210A that is attached to the proximal end portion 206A of the scanner 104 by a strain-relief element 212A. In some implementations, the scanner 104 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214A having one or more transducer elements is electrically coupled to system electronics 216A in the ultrasound machine 102. In operation, the transducer assembly 214A transmits ultrasound energy from the one or more transducer elements toward a subject and receives ultrasound echoes from the subject. The ultrasound echoes are converted into electrical signals by the transducer element(s) and electrically transmitted to the system electronics 216A in the ultrasound machine 102 for processing and generation of one or more ultrasound images. In some examples, the transmitted ultrasound energy is in the form of ultrasound pulses. The ultrasound pulses can have parameters, such as waveform, phase, amplitude, and steering angle.

Capturing ultrasound data from a subject using a transducer assembly (e.g., the transducer assembly 214A) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than a threshold megahertz (MHz) value) and/or high-frequency ultrasound (e.g., greater than the threshold MHz value). A particular frequency range to use can readily be determined based on various factors (e.g., depth of imaging, desired resolution, etc.).

In some implementations, the system electronics 216A include one or more processors (e.g., the processor(s) 106 from FIG. 1), integrated circuits, application-specific integrated circuits (ASICs), Field Programmable Gate Arrays (FPGAs), Graphics Processing Units (GPUs), and power sources to support functioning of the ultrasound machine 102. In some implementations, the ultrasound machine 102 also includes an ultrasound control subsystem 218A having one or more processors. At least one processor, FPGA, ASIC, GPU, etc. causes electrical signals to be transmitted to the transducer(s) of the scanner 104 to both emit sound waves and also receive electrical pulses from the scanner 104 that were created from the returning echoes. One or more processors, FPGAs, ASICs, GPUs, etc. process the raw data associated with the received electrical pulses and form an image that is sent to an ultrasound imaging subsystem 220A, which causes the image to be displayed (e.g., via the output module 116). In aspects, the output module 116 displays ultrasound images from the ultrasound data processed by the processor(s) of the ultrasound control subsystem 218A.

In some implementations, the ultrasound machine 102 also includes one or more user input devices (e.g., a keyboard, a cursor control device, a microphone, a camera, etc.) that input data and enable taking measurements, such as from a display from the output module 116 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, etc.) for storing the acquired ultrasound data. In aspects, the disk storage device includes the memory 108, which is local to the ultrasound machine 102. Alternatively, the memory 108 used for storing the acquisition data can be remote, such as on a remote server (e.g., medical archiver) communicatively connected to the ultrasound machine 102. In addition, the ultrasound machine 102 can include a printer that prints the image from the displayed data. To avoid obscuring the techniques described herein, some elements, such as user input devices, a disk storage device, and a printer, are not shown in FIG. 2A.

FIG. 2B illustrates another example implementation 200B of the ultrasound machine from FIG. 1. In the example implementation 200B, the scanner 104 (e.g., ultrasound scanner) is a wearable transducer 202B designed to be worn by a user. For example, the wearable transducer 202B can be a patch-based transducer. In some implementations, the wearable transducer 202B is used for imaging a constant location, such as one with periodic microbubble injections. By way of example, a mobile ultrasound monitor is used to monitor blood flow in an intensive care situation, a post-transplant patient, a trauma patient, a patient who requires minimal handling, etc.

In some implementations, the scanner 104 is wirelessly coupled to the ultrasound imaging system (e.g., the ultrasound machine 102) and communicates with the ultrasound imaging system via one or more wireless transmitters, receivers, or transceivers over a wireless connection or network (e.g., Bluetooth™, Wi-Fi™, etc.). The wearable transducer 202B is electrically coupled to system electronics 204B in the ultrasound machine 102. In operation, the wearable transducer 202B transmits ultrasound energy from one or more transducer elements toward a subject and receives ultrasound echoes from the subject. The ultrasound echoes are converted into electrical signals by the transducer element(s) and electrically transmitted to the system electronics 204B in the ultrasound machine 102 for processing and generation of one or more ultrasound images. In some examples, the transmitted ultrasound energy is in the form of ultrasound pulses. The ultrasound pulses can have parameters, such as waveform, phase, amplitude, and steering angle.

Capturing ultrasound data from a subject using a transducer assembly (e.g., the wearable transducer 202B) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than a threshold megahertz (MHz) value) and/or high-frequency ultrasound (e.g., greater than the threshold MHz value). A particular frequency range to use can readily be determined based on various factors (e.g., depth of imaging, desired resolution, etc.).

In some implementations, the system electronics 204B include one or more processors (e.g., the processor(s) 106 from FIG. 1), integrated circuits, application-specific integrated circuits (ASICs), Field Programmable Gate Arrays (FPGAs), Graphics Processing Units (GPUs), and power sources to support functioning of the ultrasound machine 102. In some implementations, the ultrasound machine 102 also includes an ultrasound control subsystem 206B having one or more processors. At least one processor, FPGA, ASIC, GPU, etc. causes electrical signals to be transmitted to the transducer(s) of the scanner 104 to both emit sound waves and also receive electrical pulses from the scanner 104 that were created from the returning echoes. One or more processors, FPGAs, ASICs, GPUs, etc. process the raw data associated with the received electrical pulses and form an image that is sent to an ultrasound imaging subsystem 208B, which causes the image to be displayed (e.g., via the output module 116). In aspects, the output module 116 displays ultrasound images from the ultrasound data processed by the processor(s) of the ultrasound control subsystem 206B.

In some implementations, the ultrasound machine 102 also includes one or more user input devices (e.g., a keyboard, a cursor control device, a microphone, a camera, etc.) that input data and enable taking measurements, such as from a display from the output module 116 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, etc.) for storing the acquired ultrasound data. In aspects, the disk storage device includes the memory 108, which is local to the ultrasound machine 102. Alternatively, the memory 108 used for storing the acquisition data can be remote, such as on a remote server (e.g., medical archiver) communicatively connected to the ultrasound machine 102. In addition, the ultrasound machine 102 can include a printer that prints the image from the displayed data. To avoid obscuring the techniques described herein, some elements, such as user input devices, a disk storage device, and a printer, are not shown in FIG. 2B.

### Example Nonlinear Contrast Imaging

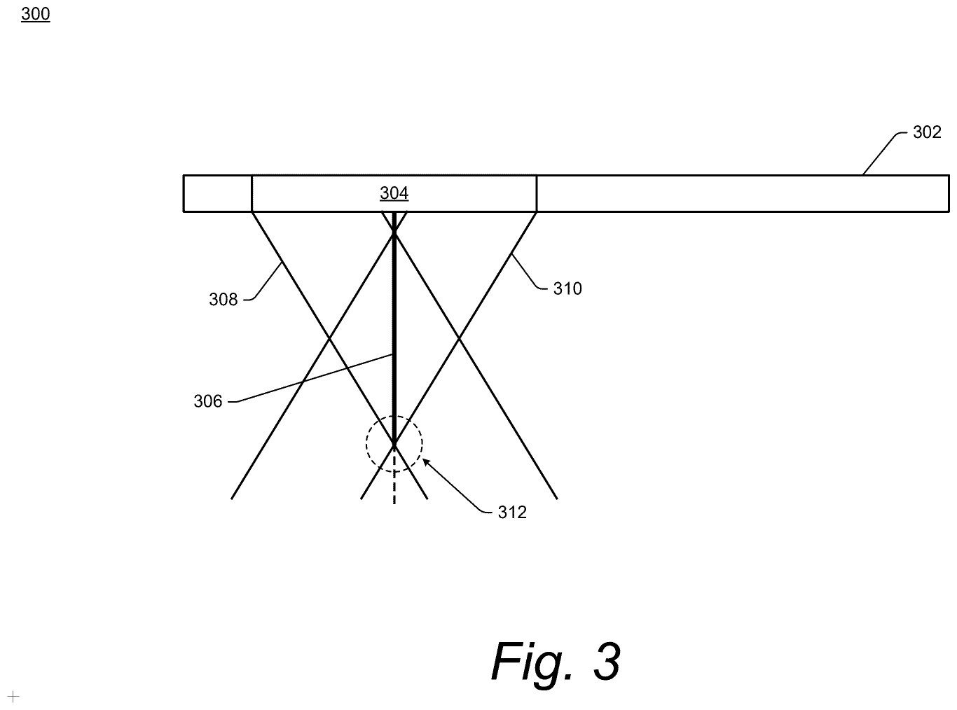

FIG. 3 illustrates an example ultrasound transducer 300 for nonlinear contrast imaging with ultrasound. The example ultrasound transducer 300 includes a multi-element array 302. For example, the multi-element array 302 can be a 256-element array. The multi-element array 302 includes an aperture 304. The aperture 304 represents a subset of elements of the multi-element array 302. For example, in a multi-element array 302 with 256 elements, the aperture 304 can have 128 elements. In aspects, the aperture 304 can be divided into at least two aperture sections by a bisector 306 (e.g., divided into equal halves, divided into ⅓ and ⅔, or other division schemes). The bisector 306 is a line in space, which bisects the aperture 304.

In some examples, the example ultrasound transducer 300 transmits a first pulse 308 and a second pulse 310. The first pulse 308 and the second pulse 310, in some examples, are steered simultaneously from each aperture section of the aperture 304 as part of a transmit event. The first pulse 308 and the second pulse 310 have a point of convergence 312 along the bisector 306. In the illustrated example, the point of convergence 312 represents the intersection of the outer parts of the beam transmitted in the first pulse 308 and the second pulse 310. The point of convergence 312 is, in some examples, an anatomy of a user within an ROI. The first pulse 308 has a first polarity and the second pulse 310 has a second polarity, the second polarity being opposite to the first polarity. Note that, while the point of convergence 312 is shown as a single point of overlap, any part of the area of overlap of the first pulse 308 and the second pulse 310 along the bisector 306 can be used for ultrasound imaging as disclosed herein, and the point of convergence 312 is shown for ease of illustration.

In some examples, the first pulse 308 and the second pulse 310 being steered simultaneously from each aperture section of the aperture 304 as part of the transmit event allows for full amplitude waveforms. In aspects, full amplitude waveforms give greater penetration characteristics than an amplitude modulation (AM) implementation. The use of full amplitude waveforms also produces a higher SNR than AM implementations. Additionally, the use of two pulses (e.g., the first pulse 308 and the second pulse 310) for imaging results in a higher frame rate than imaging with three or more pulses.

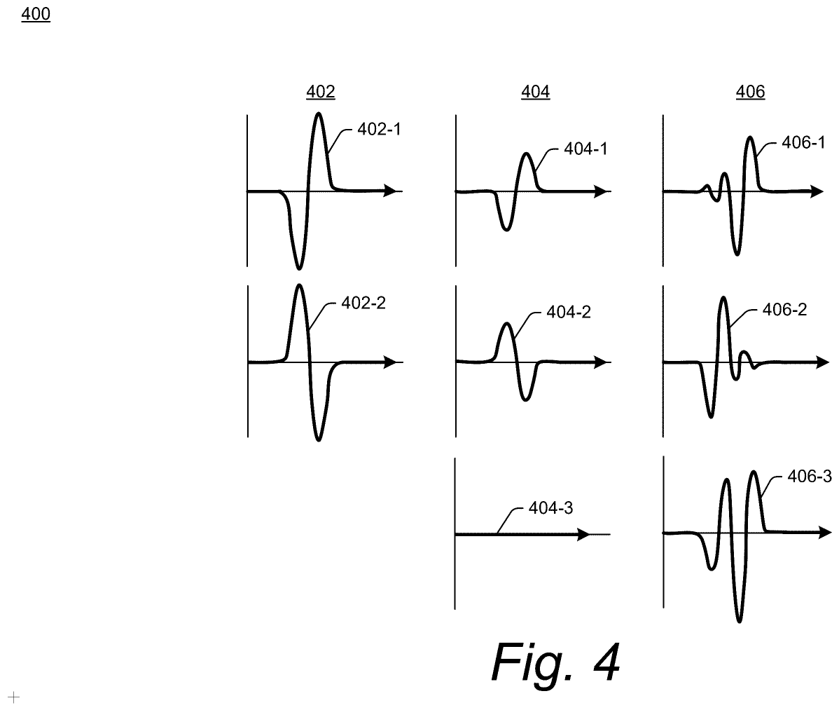

FIG. 4 illustrates an example of waveform combinations 400 for nonlinear contrast imaging with ultrasound. Initial ultrasound pulses are in a first column 402, reflected linear pulses and their combination are in a second column 404, and reflected nonlinear pulses and their combination are in a third column 406. A first incident pulse 402-1 (e.g., the first pulse 308) is shown as a first waveform. A second incident pulse 402-2 (e.g., the second pulse 310) is shown as a second waveform. As shown in Eq. 1, the second incident pulse 402-2 is a phase-inverted form of the first incident pulse 402-1.

The first incident pulse 402-1 and the second incident pulse 402-2 are transmitted at an anatomy of a user within an ROI. The anatomy reflects and/or scatters the first incident pulse 402-1 and the second incident pulse 402-2, resulting in both linear and nonlinear scattering and/or reflection components. For example, the first incident pulse 402-1 is reflected off of both a tissue, which has a linear reflection, and a contrast agent (e.g., microbubbles, including microbubbles of perfluorocarbon, nitrogen gas, sulfur hexafluoride stabilized in a phospholipid membrane, or inert gas encapsulated in a shell, autologous blood injections, or other ultrasound contrast agents), which has a nonlinear reflection.

The scattering and/or reflection of the first incident pulse 402-1 results in a first linear return signal 404-1 and a first nonlinear return signal 406-1. The scattering and/or reflecting of the second incident pulse 402-2 results in a second linear return signal 404-2 and a second nonlinear return signal 406-2. In some examples, a target anatomy being imaged is a nonlinear component of the anatomy, with linear components of the anatomy not being part of the target anatomy.

Though the first linear return signal 404-1, the first nonlinear return signal 406-1, the second linear return signal 404-2, and the second nonlinear return signal 406-2 are illustrated here as distinct signals, this should not be seen as limiting. Some or all of the return signals can be received as combinations or superpositions, including all of the return signals 404-1 through 406-2 being received as a single return signal. In some examples, a single return signal comprising the multiple return signals 404-1 through 406-2 can be broken down into constituent signals (e.g., through a Fourier decomposition). In some examples, a single return signal can be processed without further breaking down the single return signal into constituent parts, which does not affect the techniques and systems described herein.

By way of example, in contrast ultrasound imaging, microbubbles can be injected into a subject. Ultrasonic radiation directed at the subject, such as the first incident pulse 402-1 and the second incident pulse 402-2, scatters and/or reflects off of both the subject anatomy and the microbubbles. In aspects, the anatomy reflects and/or scatters linearly, as represented by the first linear return signal 404-1 and the second linear return signal 404-2. The microbubbles, in aspects, reflect and/or scatter nonlinearly, as represented by the first nonlinear return signal 406-1 and the second nonlinear return signal 406-2.

The first linear return signal 404-1 and the second linear return signal 404-2, in some examples, are combined to generate a combined linear signal 404-3. As the first incident pulse 402-1 and the second incident pulse 402-2 are phase inversions of one another, the first linear return signal 404-1 and the second linear return signal 404-2 are also phase-inverted. In some examples, this results in a combined linear signal 404-3 that is substantially close to zero. In some examples, this means that reflections from an object that reflects and/or scatters linearly (e.g., a tissue of the subject, the anatomy of the subject, etc.), are not substantially part of a total return signal. Although a linear combination has herein been described as an example, other example combination implementations are possible (e.g., time-domain combinations, frequency-domain combinations, Fourier or other orthogonal combination algorithms, etc.), thus the example of linear combinations should not be construed as limiting.

The first nonlinear return signal 406-1 and the second nonlinear return signal 406-2, in some examples, are combined to generate a combined nonlinear signal 406-3. Though the first incident pulse 402-1 and the second incident pulse 402-2 are phase inversions of one another, the nature of nonlinear scatter generally implies that the first nonlinear return signal 406-1 and the second nonlinear return signal 406-2 are not simply phase inversions of one another. In some examples, this results in the combined nonlinear signal 406-3 not being substantially close to zero. In some examples, this means that reflections from an object that reflects and/or scatters nonlinearly (e.g., a fluid of the anatomy in which the microbubbles propagate), is substantially part of a total return signal.

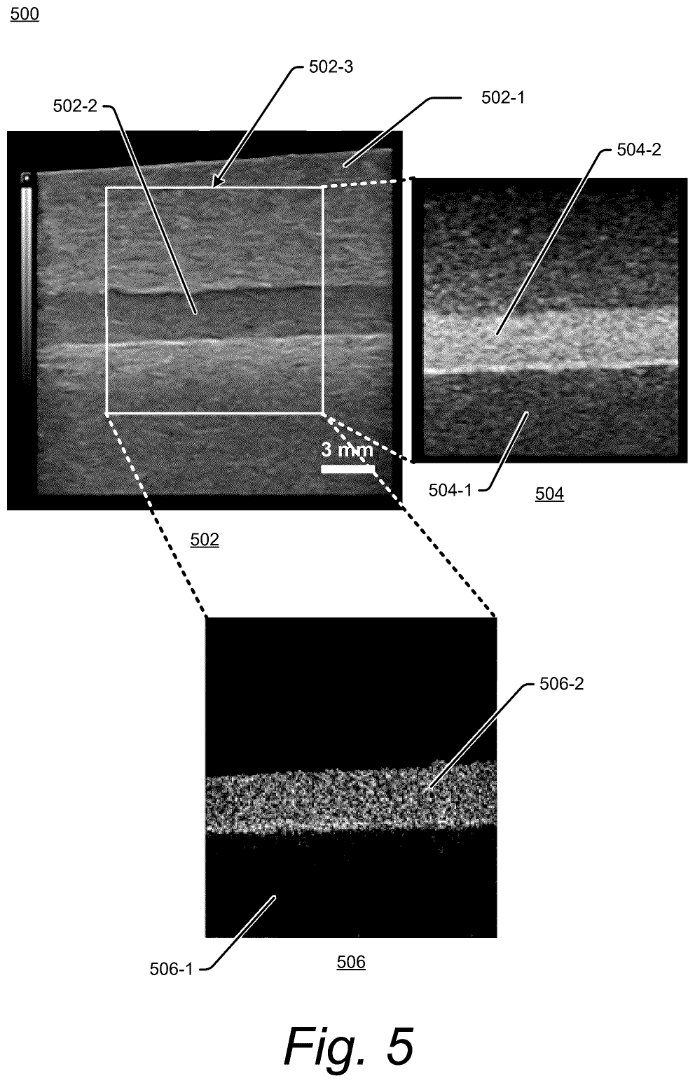

FIG. 5 illustrates example comparison results 500 using nonlinear contrast imaging with ultrasound. A first image 502 shows tissue 502-1, a vessel 502-2, and an ROI 502-3 being imaged by an ultrasound device, such as the ultrasound machine 102. A second image 504 shows the region within the ROI 502-3 imaged using a conventional focused-beam implementation of pulse inversion imaging. Portions of a tissue 504-1 above and below a vessel 504-2 are not fully suppressed on imaging. In aspects, the vessel 504-2 is the vessel 502-2, which is a target for the imaging, and the tissue 504-1 is the tissue 502-1, which is not part of the target for the imaging. The image 504 shows a poor imaging result.

A third image 506 shows nonlinear contrast imaging with ultrasound in accordance with some aspects of the present disclosure. In aspects, a vessel 506-2 is the vessel 502-2, which is a target for the imaging, and a tissue 506-1 is the tissue 502-1, which is not part of the target for the imaging. The tissue 506-1 is not imaged substantially compared to the imaging of the vessel 506-2. As outlined in the example of waveform combinations 400 of FIG. 4, the phase inversion of the first pulse 402-1 and the second pulse 402-2 enables cancelation of linear components of the ultrasound and persistence of nonlinear components. The third image 506, when compared to the second image 504, shows a high contrast difference between the vessel 506-2 and the tissue 506-1, the contrast difference being much greater than that between the vessel 504-2 and the tissue 504-1.

### Example User Interface

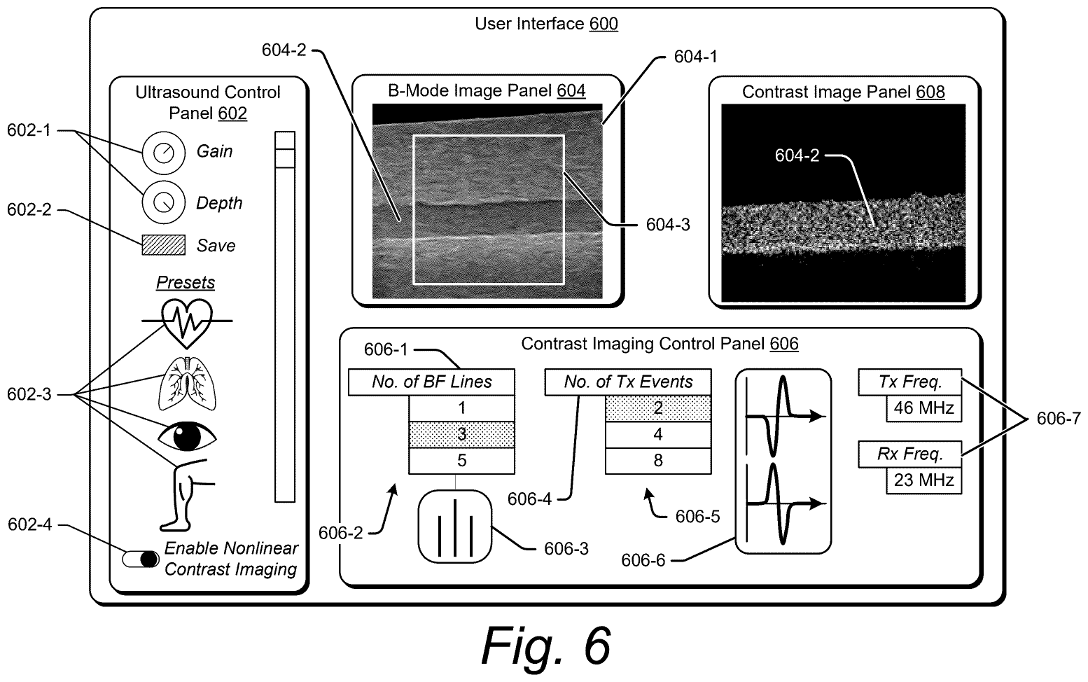

FIG. 6 illustrates an example user interface 600 for nonlinear contrast imaging with ultrasound. The example user interface 600 includes an ultrasound control panel 602, which includes ultrasound controls for adjusting gain and depth 602-1, saving an image 602-2, and selecting examination presets 602-3. The examination presets 602-3 are represented by selectable icons for a cardiac examination, a respiratory examination, an ocular examination, and a muscular-skeletal examination. The examination presets 602-3, when selected, can configure an ultrasound machine (e.g., the ultrasound machine 102) with predetermined values of gain and depth and/or other imaging parameters (e.g., beamformer settings, filter coefficients, amplitude settings, etc.). The ultrasound control panel 602 also includes a selection (e.g., an electronic rocker switch 602-4) to enable nonlinear contrast imaging.

The example user interface 600 also includes a B-mode image panel 604 for displaying any suitable type and number of ultrasound images. In some examples, the B-mode image panel 604 displays a B-mode image 604-1 that includes a blood vessel 604-2 (e.g., the first image 502 of FIG. 5), in some examples including an ROI 604-3 (e.g., the ROI 502-3 of FIG. 5). In some examples, the ROI 604-3 is drawn or otherwise selected by a user. In some examples, the ROI 604-3 is selected by the ultrasound machine, such as by a preset range, a machine-learned model, or a dynamic range. For example, based on an examination preset 602-3 selected in the ultrasound control panel 602, a machine-learned model can determine that the B-mode image 604-1 includes an anatomy consistent with the examination preset 602-3, segment the anatomy, and draw the ROI 604-3 to include at least partially the anatomy.

Responsive to the selection to enable nonlinear contrast imaging (e.g., via the rocker switch 602-4 in the ultrasound control panel 602), the example user interface 600 displays a contrast imaging control panel 606. The contrast imaging control panel 606 can display any suitable option, control, or setting to configure nonlinear contrast imaging according to the present disclosure. In some examples, the contrast imaging control panel 606 displays a selection to determine a number of beamformed lines 606-1 (e.g., to implement multi-line beamforming (as outlined later in this disclosure)). A selection of “3” lines has been made in FIG. 6 via a drop-down menu 606-2 with options of 1, 3, and 5 lines. In response to the selection of “3” via the drop-down menu 606-2, the contrast imaging control panel 606 displays a visual representation 606-3 to illustrate three lines of beamforming. The visual representation 606-3 in FIG. 6 depicts three lines and can generally include any suitable visual representation, such as a picture, an icon, an animation, a thumbnail image, and the like. In one example, the user can select a number of lines for multi-line beamforming by selecting a visual representation from among a plurality of displayed visual representations. Additionally or alternatively, the user can select the number of lines for multi-line beamforming by drawing a visual representation, such as by drawing three lines in a box to indicate to the system to perform three-line beamforming. The user can draw the lines in positions relative to a central line to indicate which lines to beamform.

The example of the contrast imaging control panel 606 in FIG. 6 also displays a selection to determine a number of transmit events 606-4 to perform nonlinear contrast imaging according to the present disclosure. For instance, a user can select via a drop-down menu 606-5 options of two, four, and eight transmit events to perform nonlinear contrast imaging. In this example, the user has selected “2” transmit events via the drop-down menu 606-5, and in response the contrast imaging control panel 606 displays a visual representation 606-6 of the number of transmit events 606-4. For instance, the contrast imaging control panel 606 displays visual representations 606-6 of transmit waveforms for the two events, including a first waveform for simultaneous transmission from both sides of a transmit aperture during a first transmit event and a second waveform for simultaneous transmission from both sides of the transmit aperture during a second transmit event. The second waveform in this example is the inverse of the first waveform. In embodiments, the user can select visual representations 606-6 of waveforms from a menu of waveforms and populate a timeline, grid, or table to (i) select a number of transmit events and (ii) indicate which waveforms are to be used for which transmit events.

The example of the contrast imaging control panel 606 in FIG. 6 also displays selections 606-7 (e.g., drop-down menus) for setting transmit and receive frequencies. In the example of FIG. 6, a user has set the transmit frequency to 46 MHz and the receive frequency to 23 MHz, indicating that the nonlinear contrast imaging is configured to receive a subharmonic of the transmit frequency. In some examples, the user also specifies the number of cycles of the transmission (Tx) pulse, or, equivalently, the fractional bandwidth (not pictured). Based on the configuration of the nonlinear contrast imaging mode set in the contrast imaging control panel 606, the user interface 600 displays a contrast image panel 608, which displays the blood vessel 604-2 indicated in the ROI 604-3 in the B-mode image panel 604 imaged according to the nonlinear contrast imaging parameters set in the contrast imaging control panel 606.

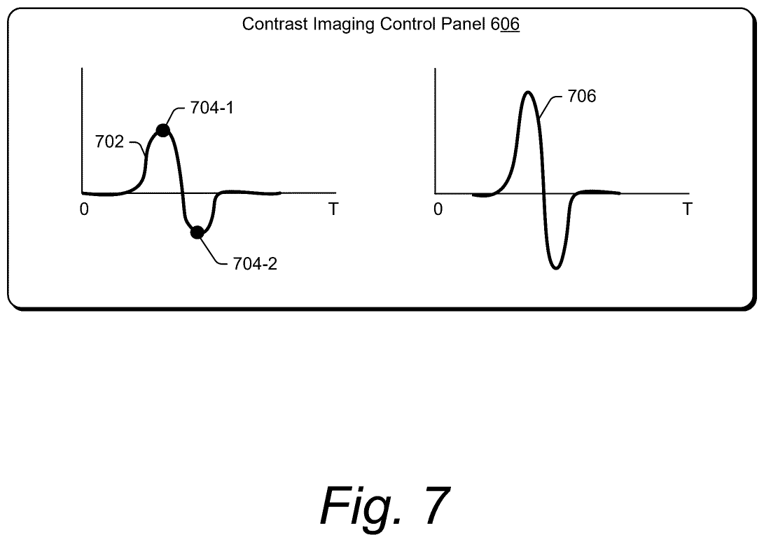

FIG. 7 illustrates an example contrast imaging control panel 606 from the user interface 600 of FIG. 6. In aspects, a user configures the system for nonlinear contrast imaging, including graphically designing a pulse (e.g., waveform) for transmission during one or more transmit events. For instance, the user selects a template pulse, assigns one or more handles to the template pulse, and then reshapes the template pulse by grabbing a handle location and dragging the handle. For example, FIG. 7 illustrates an example of the contrast imaging control panel 606 from FIG. 6, in which a user has selected a template pulse 702. For instance, the contrast imaging control panel 606 can display a plurality of template pulses (not shown for clarity), and the user selects the template pulse 702 from among the choices offered by the system. The user, in this example, has assigned two handles 704-1 and 704-2 to the template pulse 702, such as by pointing and clicking on the template pulse 702 to assign the handles 704. The user then grabs one of the handles 704, (e.g., with a cursor of a computer mouse, with a finger or stylus on a touchscreen, etc.) drags the handle 704, and deforms the template pulse 702.

In the example shown in FIG. 7, the user provides a user input that modifies the locations of the respective handles 704, which deforms the template pulse 702 via the handles 704 to cause the system to generate a pulse 706. For instance, the user provides a user input that modifies the locations of the respective handles 704, which increases the amplitude and frequency of the template pulse 702. In an example, the system automatically reshapes the template pulse 702 deformed by the user input so that the pulse 706 is implementable by the system (e.g., so that the pulse 706 can be generated by hardware and software resources of the system). The user then assigns the pulse 706 to one or more transmit events and assigns an inverse of the pulse 706 to other transmit events to perform nonlinear contrast imaging.

In some examples, frequencies that correspond with frequencies received from microbubbles are not transmitted as this could result in noticeable image clutter (e.g. a sharp change in a waveform resulting in the introduction of high frequency components in a frequency domain or Fourier representation of the pulse, asymmetric manipulations of a pulse shape, etc.). For instance, the system can enable only deformations of the template pulse 702 that do not result in frequencies and/or pulse shapes that cause image clutter. According to some examples, a “Coded Excitation” option is added to the contrast imaging control panel 606 from FIG. 6, in which the user can select to use a coded excitation, which spreads out energy over time. Additionally, in some examples, a processing scheme compatible with the concept of pulse inversion to compress signals and produce a high-SNR, high-resolution image is employed (e.g., chirps, Golay codes, etc.). Such a “Coded Excitation” option can enable the user to specify details of the code. For example, specifying a lower frequency, an upper frequency, and/or a duration for a chirp. In another example, the bit length of the coding sequence is specified for a Golay code (e.g., 4, 8, 16, etc.).

In one example, the system determines a pulse shape based on a cost function. For instance, the system can receive user inputs for weights of a cost function and then generate a pulse shape to optimize (e.g., maximize) the cost function. An example cost function is:

$${p}^{*} ( t ) - arg {max}_{{p}_{1 ( t )} \cdot {p}_{2 ( t )}} ∝ \cdot {[ {\int }_{0}^{T} ‖ F ( {p}_{1} ( t ) ) + F ( {p}_{2} ( t ) ) ‖ dt ]}^{- 1} + β \cdot [ {\int }_{0}^{T} ‖ G ( {p}_{1} ( t ) ) + G ( {p}_{2} ( t ) ) ‖ dt ] \qquad Eq . 2$$

The terms p₁(t) and p₂(t) are pulse shapes for two transmit events, the terms ∝ and β are scalars, F(⋅) is a transfer function of the linear scatterer (e.g., due to tissue and bone), and G(⋅) is a transfer function of the nonlinear scatterer (e.g., due to microbubbles or other contrast agent). The operator ∥⋅∥ denotes any suitable norm, such as an [symbol] ₁ or [symbol] ₂ norm. In an example, p₂(t) is the inverse of p₁(t) (e.g., p₁(t)=−p₂(t)). In an example, ∝ and β are user selectable. In an example, ∝∈[0,1] and β=1−α. Hence, the cost function balances the increase (e.g., maximization) of the power of the received nonlinear component while reducing (e.g., minimizing) the received linear component. Subject to the constraint that F(p₁(t))+F(p₂(t))=0 in Eq. 2 (or equivalently that p₁(t)+p₂(t)=0), the cost function can be rewritten as:

$${p}^{*} ( t ) - arg {max}_{{p}_{1 ( t )} \cdot {p}_{2 ( t )}} [ {\int }_{0}^{T} ‖ G ( {p}_{1} ( t ) ) + G ( {p}_{2} ( t ) ) ‖ dt ] \qquad Eq . 3$$

In aspects, the systems disclosed herein enable nonlinear contrast imaging on transducers due to the reduced tissue harmonic levels afforded by the system. Sampling rates can confine the imaging bandwidth (e.g., the receive bandwidth) to subharmonic frequency ranges. Subharmonics, in aspects, are enhanced by pulse inversion. In an example, a user selects a subharmonic frequency for the imaging, such as by selecting a transmit frequency F and an integer N so that a subharmonic frequency F/N is used for reception. Additionally or alternatively, a user selects a super-harmonic frequency for super-harmonic imaging. For instance, a user selects a super-harmonic frequency for the imaging, such as by selecting a transmit frequency F and an integer N so that a super-harmonic frequency N*F is used for reception.

In aspects, the systems enable modulation of artifacts if they are present in the ROI, in addition to depth range adjustment, by steering crossing transmission beams and/or pulses. In an example, saturation artifacts arising from specular reflections at tissue interfaces are sensitive to the angle of incidence of the ultrasound beam/pulse onto the interface. This angle of incidence can be adjusted by changing the steering angle of the beam without having to reposition the transducer, which can serve to alleviate the severity of an artifact when the artifact lies within the ROI.

### Example Multi-Line Nonlinear Contrast Imaging

In embodiments, the systems disclosed herein can be used for multi-line beamforming. For example, the system can generate a pulse pair aimed at a central line (e.g., the bisector 306 described above) and two adjacent lines (left and right of the central line) simultaneously, resulting in an increased frame rate. The system can reformulate beamforming equations to account for the differences in transit times for these additional lines. In an example, a user input selects a number of lines for simultaneous multi-line beamforming. Additionally or alternatively, the user input selects if the additional lines are on the left of the central line, on the right of the central line, or on both sides of the central line.

In some examples, the selection, placement, and other aspects of the additional lines are determined by a machine-learned model. For example, the machine-learned model determines the number of lines and/or the placement of lines for simultaneous multi-line beamforming based on one or more inputs, such as input parameters, the pulse pair, a user input, reflected ultrasound signals, the anatomy type in an ROI, or other inputs. In some examples, the machine-learned model determines the ROI and/or the anatomy type based on the input. In some examples, a determination to implement the multi-line beamforming is performed by the machine-learned model.

In aspects, the machine-learned model is trained using training data. The training data, in some examples, is ultrasound data based on ultrasound radiation reflected off of portions of an anatomy or a simulated anatomy. The training data may be labeled data, such as in a supervised learning algorithm, or unlabeled data, such as in an unsupervised learning algorithm. In some examples, the machine-learned model is re-trained during a scan of the anatomy. In some examples, the machine-learned model inputs and/or training inputs (e.g., the training data) are generated from an ultrasound scanner, such as the ultrasound scanner 104 of FIG. 1.

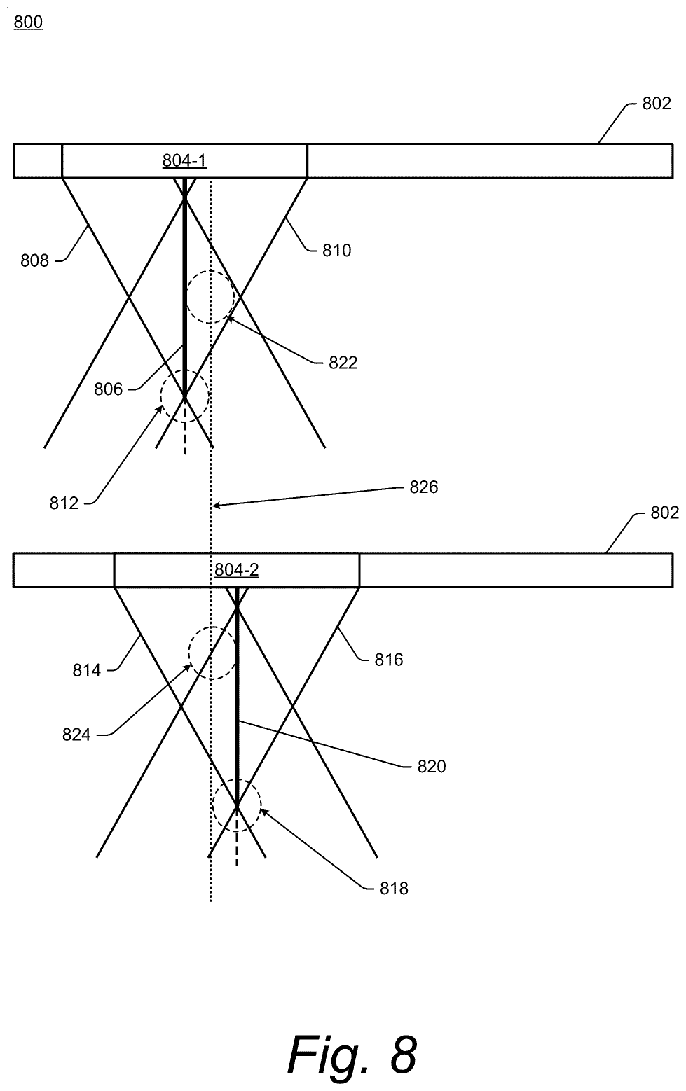

FIG. 8 illustrates an example ultrasound transducer 800 for nonlinear contrast imaging with ultrasound using multi-line scanning. The transducer 800 includes a multi-element array 802. For example, the multi-element array 802 can be a 256-element array. The multi-element array 802 includes an aperture 804. The aperture 804 represents a subset of the elements of the multi-element array 802. For example, in a multi-element array 802 with 256 elements, the aperture 804 can have 128 elements. In aspects, the aperture 804 can be divided into at least two aperture sections by a bisector 806 (e.g., divided into equal halves, divided into ⅓ and ⅔, or other division schemes). The bisector 806 is a line in space that bisects the aperture 804.

In some examples, the transducer 800 transmits a first pulse 808 and a second pulse 810 with the aperture 804-1 in a first position. The first pulse 808 and the second pulse 810, in some examples, are steered simultaneously from each aperture section of the aperture 804 as part of a transmit event. The first pulse 808 and the second pulse 810 have a point of convergence 812 along the bisector 806. The point of convergence 812 is, in some examples, an anatomy of a user within an ROI. The first pulse 808 has a first polarity and the second pulse 810 has a second polarity, the second polarity being opposite to the first polarity. Note that, while the point of convergence 812 is shown as a single point of overlap, any part of the area of overlap of the first pulse 808 and the second pulse 810 along the bisector 806 can be used for ultrasound imaging as disclosed herein, and the point of convergence 812 is shown for ease of illustration.

In some examples, the aperture 804-2 is displaced along the multi-element array 802 from the first position to a second position and transmits a third pulse 814 and a fourth pulse 816. The third pulse 814 and the fourth pulse 816, in some examples, are steered simultaneously from each aperture section of the aperture 804-2 as part of a second transmit event. The third pulse 814 and the fourth pulse 816 have a second point of convergence 818 along a second bisector 820. The second point of convergence 818 is, in some examples, the anatomy of the user within an ROI, but a different portion of the anatomy of the user than that of the point of convergence 812. The third pulse 814 has the first polarity and the fourth pulse 816 has the second polarity.

Note that, while the second point of convergence 818 is shown as a single point of overlap, any part of the area of overlap of the third pulse 814 and the fourth pulse 816 along the second bisector 820 can be used for ultrasound imaging as disclosed herein, and the second point of convergence 818 is shown for ease of illustration. Additionally or alternately, though the aperture 804-2 is shown as being displaced along the multi-element array 802 to the second position, this need not be the case. Similar results can be obtained by changing the steering angles for the third pulse 814 and the fourth pulse 816 without displacing the aperture 804-1 from the first position. Other, equivalent methods of transmitting the third pulse 814 and the fourth pulse 816 may be performed without altering the methodology used, and thus the example of displacing the aperture 804-2 to the second position along the multi-element array 802 should be seen as illustrative and not limiting.

Note that, in the example ultrasound transducer 800, a point 822 and a point 824 lie along a third bisector 826. Thus, the points 822 and 824 are located in a same point in space and are a same point in the anatomy of the user. The third bisector 826 is thus a bisector for a convergence between the second pulse 810 and the third pulse 814. In some examples, the first pulse 808 and the second pulse 810 represent a first transmit event at a first time and the third pulse 814 and the fourth pulse 816 represent a second transmit event at a second time, the second time after the first time. This example illustrates the second pulse 810 and the third pulse 814 incident upon the third bisector 826 at different times.

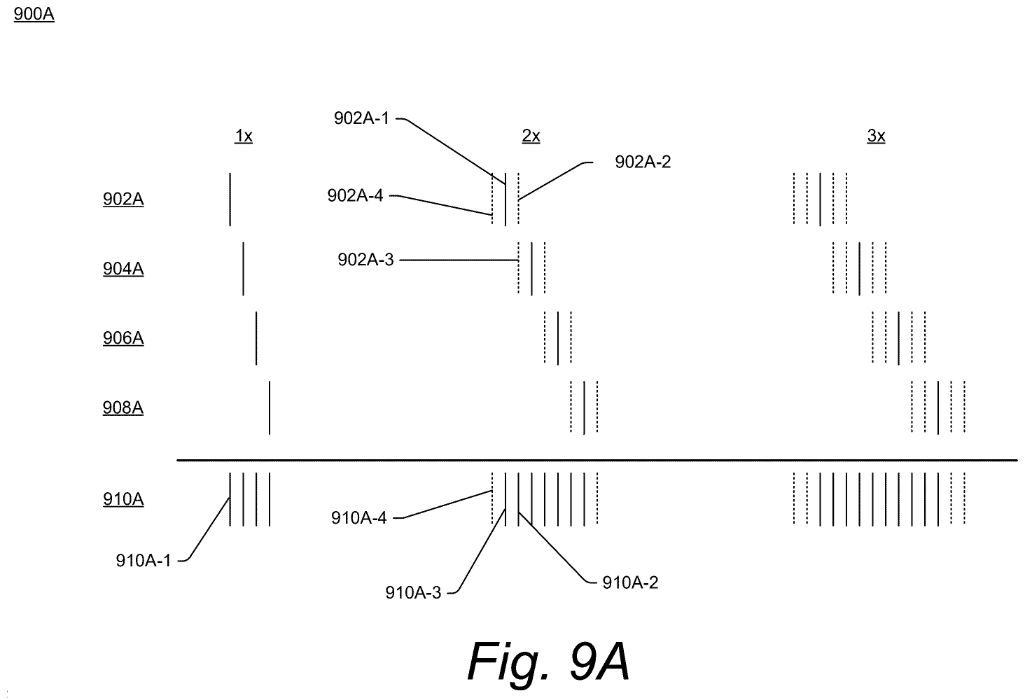

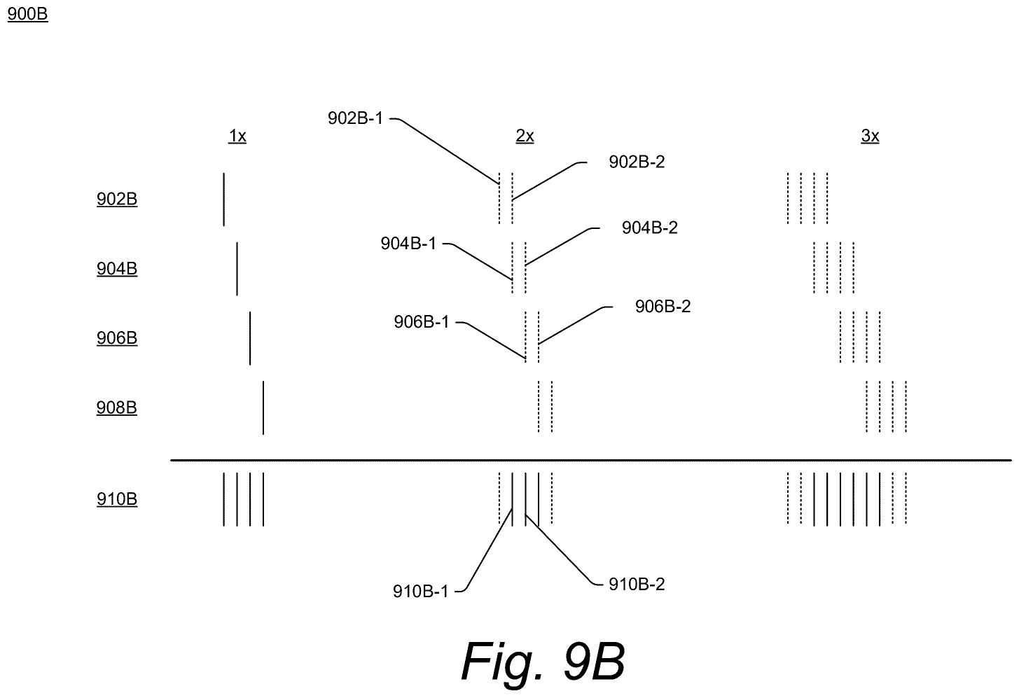

FIG. 9A illustrates an outline of a multi-line embodiment 900A for nonlinear contrast imaging with ultrasound. Multiple transmit events are shown, including a first transmit event in row 902A, a second transmit event in row 904A, a third transmit event in row 906A, and a fourth transmit event in row 908A. Each transmit event in rows 902A through 908A can have buffer lines on either side of a main solid line, with the number of solid lines plus buffer lines on one side indicated by the column headers 1x, 2x, and 3x. Converged reception lines in row 910A represent return values from the transmit events in rows 902A through 908A. Transmit events are referred to by their row and column number, separated by a hyphen; for example, the first transmit event in row 902A, column 1x, is referred to as the transmit event 902A-1x. Following this nomenclature, the converged reception lines in row 910A are referred to by their row and column number, separated by a hyphen. For example, the combined reception lines for row 910A column 1x are referred to as the converged reception lines 910A-1x. Each row 902A through 908A and column 1x through 3x represents a separate transmit event, although events in the same column are in sequence. For example, the transmit event 902A-2x is followed by the transmit event 904A-2x, which in turn is followed by the transmit event 906A-2x. The converged reception lines 910A-2x show the combination of the transmit events in the 2x column, as outlined below.

The transmit event 902A-1x shows a single convergence of two ultrasound pulses, such as the convergence of the first pulse 808 and the second pulse 810 at the bisector 806 of FIG. 8. The converged reception lines of row 910A illustrate a result of a single line of reception, represented by the first line 910A-1 of the converged reception lines 910A-1x. Subsequently, the transmit events 904A-1x, 906A-1x, and 908A-1x result in the full converged reception lines of 910A-1x. Note that, in the example multi-line embodiment 900A, two pulses from a transmit event, such as the transmit event 902A-1x, are required for a full return signal to result in a full line of the converged reception lines in row 910A. In the transmit events 902A-1x through 908A-1x, all lines are from two pulses and, thus, the converged reception lines 910A-1x are shown as solid lines with one line per transmit event.

In the 2x column, the transmit events 902A-2x through 908A-2x have a dashed line on either side of a solid line 902A-1. The solid line 902A-1 represents a two-pulse transmission, such as the convergence of the first pulse 808 and the second pulse 810 at the bisector 806 of FIG. 8. The dashed lines represent a single pulse transmission portion, which can be combined with another single pulse transmission portion from a later time at a same location. Combinations of pulses follow the methodology of phase-inverted pulses being combined, such as outlined above for FIG. 4. For example, consider a right-side dashed line 902A-2 of the transmit event 902A-2x. This is analogous to the second pulse 810 of FIG. 8 incident at the point 822 along the third bisector 826. Consider a left-side dashed line 902A-3 of the transmit event 904A-2x, which lines up with the right-side dashed line 902A-2 of the transmit event 902A-2x. This is analogous to the third pulse 814 of FIG. 8 incident at the point 824 along the third bisector 826, which is substantially the same point as the point 822.

In such an example, the second pulse 810 and the third pulse 814 are phase-inverted. This results in a second solid line 910A-2 of the converged reception lines 910A-2x, with a first solid line 910A-3 of the converged reception lines 910A-2x being the solid line of the transmit event 902A-2x and a first dashed line 910A-4 of the converged reception lines 910A-2x being a first dashed line 902A-4 of the transmit event 902A-2x. For example, the first dashed line 902A-4 of the transmit event 902A-2x can be a non-combined signal from the first pulse 808 of FIG. 8 and, as there is not a phase-inversed pulse at a point to the left of the bisector 806 with which to combine the first pulse 808, there is no corresponding solid line in the converged reception lines 910A-2x for the dashed line 902A-4.

A similar methodology using the remaining transmit events 906A-2x through 908A-2x results in the converged reception lines 910A-2x having seven solid lines, as contrasted with only four solid lines in the converged reception lines 910A-3x. Using the same methodology outlined above, the transmit events 902A-3x through 908A-3x, where there are two dashed lines on either side of the solid line for each of the transmit events 902A-3x through 908A-3x, result in the converged reception lines 910A-3x, which have ten total solid lines.

The number of total reception lines in the converged reception lines in row 910A is dependent on the number of events processed and the number of solid lines plus buffered lines on either side of the solid lines per transmit event in rows 902A through 908A. For example, the converged reception lines 910A-2x have seven solid lines over four transmit events using a solid line with a single dashed line on either side of the solid line. This can be represented mathematically as:

$${R}_{4}^{2} = 7 \qquad Eq . 4$$

In Eq. 4,

$${R}_{4}^{2}$$

represents the number of solid reception lines given four transmit events with one buffer line on either side of the solid line (given by the “2” in

$${R}_{4}^{2} ) .$$

This can be represented more generally as:

$${R}_{n}^{t} = n + \frac{t}{2} ( n - 1 ) \qquad Eq . 5$$

In Eq. 5,

$${R}_{n}^{t}$$

represents the number of solid reception lines given n transmit events and t buffer lines. Using Eq. 5,

$${R}_{4}^{2} = 4 + 1 ( 4 - 1 ) = 7$$

is recovered. The ten solid lines of the converged reception lines 910A-3x via

$${R}_{4}^{2} = 4 + 2 ( 4 - 1 ) = 10$$

are similarly recovered. Transmit events after the first transmit event 902A have t/2 dashed lines available for combination in addition to the solid line from the nth event, which is given mathematically by Eq. 5.

Combining the dashed lines, as in Eq. 5, enables increased resolution over simply using the solid lines (e.g., the converged reception lines 910A-1x). For example, using a combination of the second pulse 810 with the third pulse 814 along the third bisector 826 in addition to (i) the combination of the first pulse 808 and the second pulse 810 along the bisector 806 and (ii) the combination of the third pulse 814 and the fourth pulse 816 along the second bisector 820 results in, per the converged reception lines 910A-2x and considering only the transmit events 902A-2x and 906A-2x, three solid lines from two events instead of only two solid lines.

In some examples, this methodology results in a significant frame rate increase. Taking the lines of the converged reception lines in row 910A as a measure positively corresponding monotonically with frame rate, a 1× scheme nets n solid lines. For example, an ultrasound imaging with n=200 transmit events corresponds to a solid line count of 200 (e.g., using no multi-line processing) for the final, converged lines (e.g., the converged reception lines 910A-1x as they would appear after processing 200 transmit events). Consider an example where there are t=6 buffer lines. In the same n=200 transmit events, utilizing 6 buffer lines would result in 797 solid lines, showing an almost 4× increase. For a large n compared to t, Eq. 5 can be reformatted and approximated as:

$${R}_{n}^{t} = n ( 1 + \frac{t}{2} ) - \frac{t}{2} \approx n ( 1 + \frac{t}{2} ) \qquad Eq . 6$$

According to some examples, a central line (e.g., the solid lines 902A-1, 902A-3, etc.) will be aligned with the center of incident pulses (e.g., the two ultrasound pulses) while neighboring lines (e.g., the dashed line 902A-2, the dashed line 902A-4, etc.) are off-center. According to some examples, this may cause the central line to have an increased resolution, SNR, or other characteristic compared to the neighboring lines. In such examples, only the neighboring lines are taken into consideration during signal reconstruction and/or imaging, which avoids potential aberrations in the signal netting a more consistent and better-aligned multi-line implementation.

FIG. 9B illustrates an outline of a multi-line embodiment 900B without a central line for nonlinear contrast imaging with ultrasound. In some examples, the imaging lines are combined across a receiving aperture using only the neighboring lines, resulting in overlapping off-center lines and a centered beamformed line. As the central lines are not imaged, the increase in potential image quality is at the expense of absolute frame rate. Consider transmit events 902B-2x, 904B-2x, 906B-2x, and 908B-2x resulting in converged receptions lines 910B-2x. Two solid lines 910B-1 and 910B-2 represent converged receptions for the transmit events 902B-2x through 906B-2x. Dashed lines 902B-2 and 904B-1 combine to generate 910B-1 and dashed lines 904B-2 and 906B-1 combine to generate 910B-2.

### Example Methods

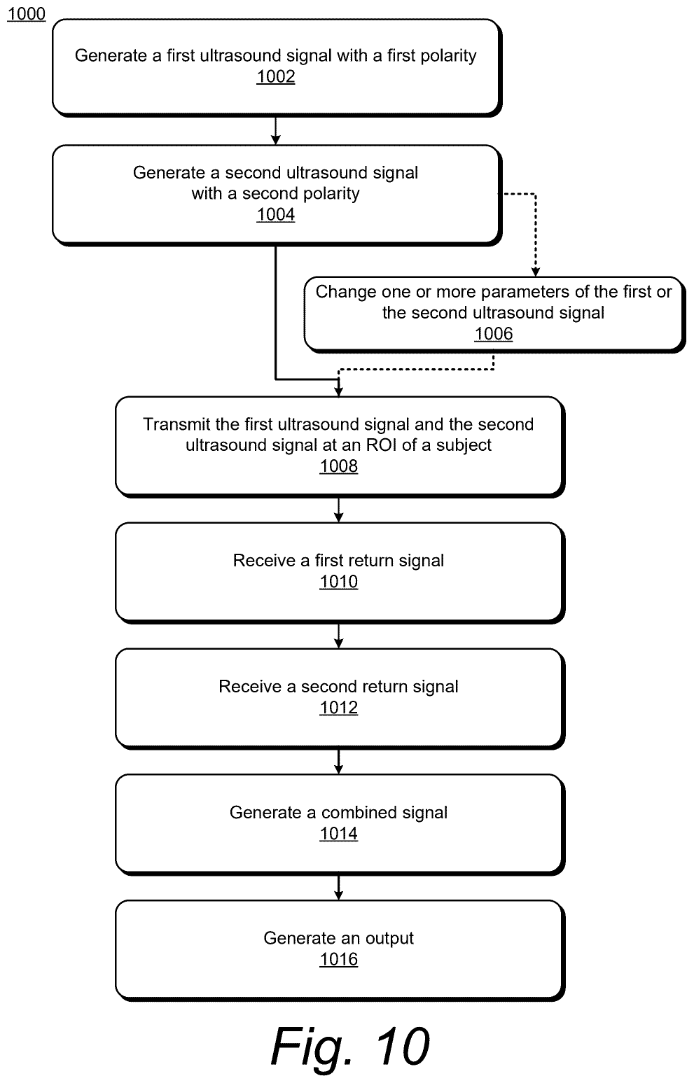

FIG. 10 outlines a method 1000 for nonlinear contrast imaging with ultrasound. The method 1000 is shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example environment 100 of FIG. 1 or to entities or processes as detailed in FIGS. 2-9, reference to which is made for example only. The techniques are not limited to performance by one entity or multiple entities operating on one device. The method 1000 can be performed by an ultrasound machine, such as the ultrasound machine 102 described herein.

At 1002, a first ultrasound signal with a first polarity is generated. In some examples, the first ultrasound signal is a first pulse. In some examples, the first pulse is steered at a first steering angle, the first steering angle being an angle relative to a face surface of an ultrasound transducer (e.g., the ultrasound transducer 300).

At 1004, a second ultrasound signal with a second polarity is generated. The second polarity can be the inverse of the first polarity. In aspects, the second ultrasound signal is a phase-inverted version of the first ultrasound signal (e.g., Eq. 1). For example, a plane wave can take the general form:

$$ψ = A ( {e}^{i ( \vec{k} \cdot \vec{r} + ω \cdot t + ϕ )} + {e}^{- i ( \vec{k} \cdot \vec{r} + ω \cdot t + ϕ )} ) \qquad Eq . 7$$

The term ψ in Eq. 7 is a wave with amplitude A, wave number k, angular frequency ω, and phase ϕ. For a wave equation for the first ultrasound signal represented by ψ₁ and with an angular frequency ϕ₁, a wave equation for the second ultrasound signal represented by ψ₂ would be the same as ψ₁ but with an angular frequency ϕ₂=ϕ₁+π (or other inversions techniques, e.g., Eq. 1). In some examples, the second ultrasound signal is a second pulse. In some examples, the second pulse is steered at a second steering angle, the second steering angle being an angle relative to the face surface of the ultrasound transducer (e.g., the ultrasound transducer 300). In aspects, the second steering angle is configured such that the first pulse and the second pulse converge at a point along a bisector of a portion of the transducer used to produce the first and the second pulses. In some examples, the first pulse and the second pulse are full-amplitude pulses. At 1006, in some examples, a change is made to one or more parameters of the first ultrasound signal or the second ultrasound signal. In some examples, the one or more parameters include a phase of the first pulse or the second pulse, an amplitude of the first pulse or the second pulse, a frequency of the first pulse or the second pulse, or a waveform of the first pulse or the second pulse. The changing of the one or more parameters, in some examples, is responsive to a user input. In some examples, the changing of the one or more parameters is performed automatically. For example, the changing of the one or more parameters can be based on a machine-learned model. The machine-learned model can be, for instance, a feature-extracting model, such as a convolutional neural network (CNN), a model trained by supervised learning, a model trained by unsupervised learning, or any other form of machine-learned model known to a person of ordinary skill in the art.

At 1008, the first ultrasound signal and the second ultrasound signal are transmitted at an ROI of a subject. In some examples, the transmitting of the first ultrasound signal and the second ultrasound signal at the ROI includes determining a coverage area of the transmission of the first ultrasound signal and the second ultrasound signal. The transmitting of the first ultrasound signal and the second ultrasound signal at the ROI, in some examples, further includes determining a bisector of the coverage area. In aspects, the first ultrasound signal and the second ultrasound signal are configured to converge at a point on the bisector. In some examples, the transmission of the first ultrasound signal and the second ultrasound signal occur at a same time.

At 1010, a first return signal is received. In aspects, the first return signal is based on the first ultrasound signal reflecting from the ROI. In some examples, the first return signal includes a first linear component and a first nonlinear component. In some examples, the first linear component is related to scattering and/or reflecting of the first ultrasound signal from one or more linear reflectors (e.g., portions of an anatomy of the subject) within the ROI, which scatter and/or reflect in a linear manner. Similarly, in some examples, the first nonlinear component is related to scattering and/or reflecting of the first ultrasound signal from one or more nonlinear reflectors within the ROI (e.g., microbubbles), which scatter and/or reflect in a nonlinear manner.

At 1012, a second return signal is received. In aspects, the second return signal is based on the second ultrasound signal reflecting from the ROI. In some examples, the second return signal includes a second linear component and a second nonlinear component. In some examples, the second linear component is related to scattering and/or reflecting of the second ultrasound signal from the one or more linear reflectors (e.g., portions of the anatomy of the subject) within the ROI, which scatter and/or reflect in a linear manner. Similarly, in some examples, the second nonlinear component is related to scattering and/or reflecting of the second ultrasound signal from the one or more nonlinear reflectors (e.g., microbubbles or other contrast agents) within the ROI, which scatter and/or reflect in a nonlinear manner. In aspects, the first linear component is substantially opposite to the second linear component due to the phase inversion of ψ₂ vs. ψ₁. In aspects, the changing of the one or more parameters in 1006 is based on at least one of the first return signal or the second return signal.

At 1014, a combined signal is generated. In aspects, the combined signal is based on the first return signal and the second return signal. The generation of the combined signal, in some examples, represents a beamforming of the first pulse and the second pulse at the ROI. In some examples, the generating of the combined signal is based on a linear combination of the first return signal and the second return signal. In examples where the first linear component is substantially opposite to the second linear component due to the phase inversion of ψ₂ vs. ψ₁, the generation of the combined signal does not substantially contain information about the first linear component and the second linear component but retains information about the first nonlinear component and the second nonlinear component. In some examples, the generating of the combined signal is the result of a summing of the first return signal and the second return signal.

At 1016, an output is generated. In aspects, the output is based on the combined signal. In some examples, the output is an image or a component of the image, the image configured to contain information pertaining to, for example, a target anatomy in the ROI. The image can be based on the combined signal. In some examples, the output is configured to be displayed on a display element (e.g., the display 122). In some examples, the output is a value, such as a property of the target anatomy in the ROI.

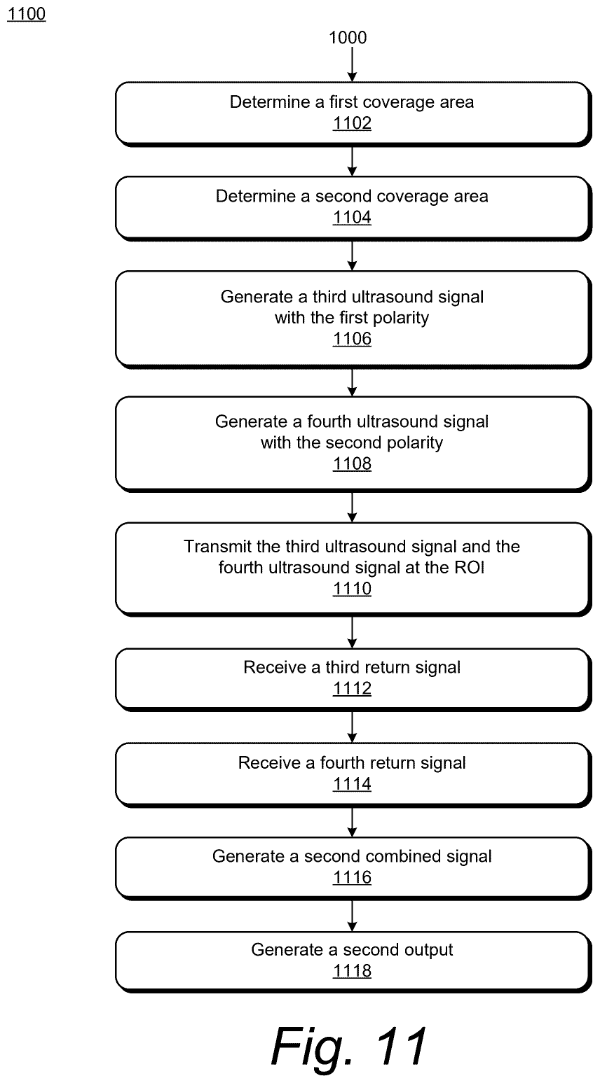

FIG. 11 outlines a method 1100 for multi-line nonlinear contrast imaging with ultrasound. The method 1100 is shown as a set of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example environment 100 of FIG. 1 or to entities or processes as detailed in FIGS. 2-10, reference to which is made for example only. The techniques are not limited to performance by one entity or multiple entities operating on one device. The method 1100 continues from the method 1000.

At 1102, a first coverage area is determined. The first coverage area, in aspects, is an area within an ROI where the first ultrasound signal and the second ultrasound signal are incident. At 1104, a second coverage area is determined. The second coverage area, in aspects, is an area within the ROI where a transmission of a third ultrasound signal and a fourth ultrasound signal are incident, the second coverage area overlapping the first coverage area at least in part.

At 1106, a third ultrasound signal is generated with the first polarity. In some examples, the third ultrasound signal is a third pulse. In some examples, the third pulse is steered at a third steering angle, the third steering angle being an angle relative to the face surface of the ultrasound transducer (e.g., the ultrasound transducer 300). In aspects, the third steering angle is configured such that the third pulse and the second pulse converge at a second point along a second bisector of a portion of the transducer used to produce the third and the fourth pulses.

At 1108, a fourth ultrasound signal is generated with the second polarity. In some examples, the fourth ultrasound signal is a fourth pulse. In some examples, the fourth pulse is steered at a fourth steering angle, the fourth steering angle being an angle relative to the face surface of the ultrasound transducer (e.g., the ultrasound transducer 300). In aspects, the fourth steering angle is configured such that the third pulse and the fourth pulse converge at a third point along the second bisector of the portion of the transducer used to produce the third and the fourth pulses.

At 1110, the third ultrasound signal and the fourth ultrasound signal are transmitted at the ROI. In some examples, the transmitting the third ultrasound signal and the fourth ultrasound signal at the ROI includes the determining of the second coverage area. The transmitting the first ultrasound signal and the second ultrasound signal at the ROI, in some examples, further includes determining a bisector of the coverage area. In aspects, the first ultrasound signal and the second ultrasound signal are configured to converge at a point on the bisector.

At 1112, a third return signal is received. In aspects, the third return signal is based on the third ultrasound signal reflecting from the ROI. In some examples, the third return signal includes a third linear component and a third nonlinear component. In some examples, the third linear component is related to scattering and/or reflecting of the third ultrasound signal from the one or more portions of the anatomy of the subject within the ROI, which scatters and/or reflects in a linear manner. Similarly, in some examples, the third nonlinear component is related to scattering and/or reflecting of the third ultrasound signal from the one or more portions of the anatomy of the subject within the ROI, which scatters and/or reflects in a nonlinear manner.

At 1114, a fourth return signal is received. In aspects, the fourth return signal is based on the fourth ultrasound signal reflecting from the ROI. In some examples, the fourth return signal includes a fourth linear component and a fourth nonlinear component. In some examples, the fourth linear component is related to scattering and/or reflecting of the fourth ultrasound signal from the one or more portions of the anatomy of the subject within the ROI, which scatters and/or reflects in a linear manner. Similarly, in some examples, the fourth nonlinear component is related to scattering and/or reflecting of the fourth ultrasound signal from the one or more portions of the anatomy of the subject within the ROI, which scatters and/or reflects in a nonlinear manner.

At 1116, a second combined signal is generated. In aspects, the second combined signal is based on the second return signal and the third return signal. For instance, the second combined signal can include a summation of the second return signal and the third return signal. The generation of the second combined signal, in some examples, represents a beamforming of the second pulse and the third pulse at the ROI. In some examples, the generating of the second combined signal is based on a linear combination of the second return signal and the third return signal. In examples where the second linear component is substantially opposite to the third linear component due to the phase inversion of a third wave function ψ₃ vs. ψ₂, the generation of the second combined signal does not substantially contain information about the second linear component and the third linear component but retains information about the second nonlinear component and the third nonlinear component (e.g., information about the one or more nonlinear scatterers, such as microbubbles or another contrast agent).

At 1118, a second output is generated. In aspects, the second output is based on the second combined signal. In some examples, the second output is a second image or a component of the second image, the second image configured to contain information pertaining to a second target anatomy in the ROI. In some examples, the second output is configured to be displayed on the display element (e.g., the display 122). In some examples, the second output is a second value, such as the property of the target anatomy in the ROI.

### ADDITIONAL EXAMPLES

The following are additional examples of the described devices and methods for nonlinear contrast imaging with ultrasound.

Example 1. An ultrasound device including an ultrasound scanner, the ultrasound scanner configured to generate a first ultrasound signal with a first polarity and a second ultrasound signal with a second polarity, the second polarity being an inverse of the first polarity. The ultrasound scanner is further configured to transmit the first ultrasound signal and the second ultrasound signal at a region of interest (ROI) of a subject, receive a first return signal based on the first ultrasound signal reflecting from the ROI, and receive a second return signal based on the second ultrasound signal reflecting from the ROI. The ultrasound device further includes one or more processors and a memory, the memory storing instructions that, when executed by the one or more processors, cause the one or more processors to generate a combined signal based on the first return signal and the second return signal and an output based on the combined signal.

Example 2. The ultrasound device of example 1, wherein the transmission of the first ultrasound signal and the second ultrasound signal occur at a same time.

Example 3. The ultrasound device of example 1, where the first ultrasound signal includes a first pulse and the second ultrasound signal includes a second pulse.

Example 4. The ultrasound device of example 3, where the generation of the combined signal represents a beamforming of the first pulse and the second pulse at the ROI.

Example 5. The ultrasound device of example 3, where the instructions further cause the one or more processors to change one or more parameters of the first ultrasound signal or the second ultrasound signal.

Example 6. The ultrasound device of example 5, where the changing of the one or more parameters is based on at least one of the first return signal or the second return signal.

Example 7. The ultrasound device of example 5, where the one or more parameters include a phase of the first pulse or the second pulse, an amplitude of the first pulse or the second pulse, a frequency of the first pulse or the second pulse, or a waveform of the first pulse or the second pulse.

Example 8. The ultrasound device of example 5, where the changing of the one or more parameters is responsive to a user input.

Example 9. The ultrasound device of example 5, where the changing of the one or more parameters is based on a machine-learned model.

Example 10. The ultrasound device of example 1, where the first return signal includes a first linear component and a first nonlinear component, the second return signal includes a second linear component and a second nonlinear component, and the generating of the combined signal is based on a linear combination of the first return signal and the second return signal.

Example 11. The ultrasound device of example 10, where the first linear component is substantially opposite to the second linear component.

Example 12. The ultrasound device of example 10, wherein the first nonlinear component and the second nonlinear component are based on nonlinear reflections from a contrast agent within the ROI.

Example 13. The ultrasound device of example 3, wherein the first pulse and the second pulse are full-amplitude pulses.

Example 14: The ultrasound device example 1, wherein the generating of the combined signal is the result of a summing of the first return signal and the second return signal.

Example 15. The ultrasound device of example 1, where the configuring of the ultrasound scanner to transmit the first ultrasound signal and the second ultrasound signal at the ROI includes determining a coverage area of the transmission of the first ultrasound signal and the second ultrasound signal and a bisector of the coverage area, and the first ultrasound signal and the second ultrasound signal are configured to converge at a point on the bisector.

Example 16. The ultrasound device of example 1, where the ultrasound scanner is further configured to generate a third ultrasound signal with the first polarity, generate a fourth ultrasound signal with the second polarity, transmit the third ultrasound signal and the fourth ultrasound signal at the ROI, receive a third return signal based on the third ultrasound signal reflecting from the ROI, and receive a fourth return signal based on the fourth ultrasound signal reflecting from the ROI. The instructions further cause the one or more processors to generate a second combined signal based on the third return signal and the second return signal and a second output based on the second combined signal.

Example 17. The ultrasound device of example 16, where the ultrasound scanner is further configured to determine a first coverage area of the transmission of the first ultrasound signal and the second ultrasound signal and a second coverage area of the transmission of the third ultrasound signal and the fourth ultrasound signal, the second coverage area overlapping the first coverage area at least in part.

Example 18. A method for nonlinear contrast imaging with ultrasound, the method including generating, by an ultrasound scanner, a first ultrasound signal with a first polarity and a second ultrasound signal with a second polarity, the second polarity being an inverse of the first polarity. The method further includes transmitting, by the ultrasound scanner, the first ultrasound signal and the second ultrasound signal at a region of interest (ROI) of a subject, receiving, by the ultrasound scanner, a first return signal based on the first ultrasound signal reflecting from the ROI and a second return signal based on the second ultrasound signal reflecting from the ROI, and generating, by one or more processors, a combined signal based on the first return signal and the second return signal and an output based on the combined signal.

Example 19. The method of example 18, wherein the transmission of the first ultrasound signal and the second ultrasound signal occur at a same time.

Example 20. The method of example 18, where the first ultrasound signal comprises a first pulse and the second ultrasound signal comprises a second pulse.

Example 21. The method of example 20, where the generation of the combined signal represents a beamforming of the first pulse and the second pulse at the ROI.

Example 22. The method of example 20, further comprising changing, by the one or more processors, one or more parameters of the first ultrasound signal or the second ultrasound signal.

Example 23. The method of example 22, where the changing of the one or more parameters is based on at least one of the first return signal or the second return signal.

Example 24. The method of example 22, where the one or more parameters include a phase of the first pulse or the second pulse, an amplitude of the first pulse or the second pulse, a frequency of the first pulse or the second pulse, or a waveform of the first pulse or the second pulse.

Example 25. The method of example 22, where the changing of the one or more parameters is responsive to a user input.

Example 26. The method of example 22, where the changing of the one or more parameters is based on a machine-learned model.

Example 27. The method of example 18, where the first return signal includes a first linear component and a first nonlinear component, the second return signal includes a second linear component and a second nonlinear component, and the generating of the combined signal is based on a linear combination of the first return signal and the second return signal.

Example 28. The method of example 27, where the first linear component is substantially opposite to the second linear component.

Example 29. The method of example 27, wherein the first nonlinear component and the second nonlinear component are based on nonlinear reflections from a contrast agent within the ROI.

Example 30. The method of example 20, wherein the first pulse and the second pulse are full-amplitude pulses.

Example 31: The method example 18, wherein the generating of the combined signal is the result of a summing of the first return signal and the second return signal.

Example 32. The method of example 18, where the transmitting the first ultrasound signal and the second ultrasound signal at the ROI includes determining, by the ultrasound scanner, a coverage area of the transmission of the first ultrasound signal and the second ultrasound signal and a bisector of the coverage area, where the first ultrasound signal and the second ultrasound signal are configured to converge at a point on the bisector.

Example 33. The method of example 18, further including generating, by the ultrasound scanner, a third ultrasound signal with the first polarity and a fourth ultrasound signal with the second polarity and transmitting, by the ultrasound scanner, the third ultrasound signal and the fourth ultrasound signal at the ROI. The method further includes receiving, by the ultrasound scanner, a third return signal based on the third ultrasound signal reflecting from the ROI and a fourth return signal based on the fourth ultrasound signal reflecting from the ROI. The method further includes generating, by the one or more processors, a second combined signal based on the third return signal and the second return signal and a second output based on the second combined signal.

Example 34. The method of example 33, further including determining, by the ultrasound scanner, a first coverage area of the transmission of the first ultrasound signal and the second ultrasound signal and a second coverage area of the transmission of the third ultrasound signal and the fourth ultrasound signal, the second coverage area overlapping the first coverage area at least in part.

Example 35. A user interface for an ultrasound device, the user interface configured to cause an ultrasound scanner of the ultrasound device to generate a first ultrasound signal with a first polarity, generate a second ultrasound signal with a second polarity, the second polarity being an inverse of the first polarity, transmit the first ultrasound signal and the second ultrasound signal at a region of interest (ROI) of a subject, receive a first return signal based on the first ultrasound signal reflecting from the ROI, and receive a second return signal based on the second ultrasound signal reflecting from the ROI. the user interface is further configured to cause one or more processors of the ultrasound device to generate a combined signal based on the first return signal and the second return signal and an output based on the combined signal.

Example 36. The user interface of example 35, wherein the transmission of the first ultrasound signal and the second ultrasound signal occur at a same time.

Example 37. The user interface of example 35, where the first ultrasound signal includes a first pulse and the second ultrasound signal includes a second pulse.

Example 38. The user interface of example 37, where the generation of the combined signal represents a beamforming of the first pulse and the second pulse at the ROI.

Example 39. The user interface of example 37, where the user interface is further configured to cause the one or more processors to change one or more parameters of the first ultrasound signal or the second ultrasound signal.

Example 40. The user interface of example 39, where the changing of the one or more parameters is based on at least one of the first return signal or the second return signal.

Example 41. The user interface of example 39, where the one or more parameters include a phase of the first pulse or the second pulse, an amplitude of the first pulse or the second pulse, a frequency of the first pulse or the second pulse, or a waveform of the first pulse or the second pulse.

Example 42. The user interface of example 39, where the changing of the one or more parameters is responsive to a user input.

Example 43. The user interface of example 39, where the changing of the one or more parameters is based on a machine-learned model.

Example 44. The user interface of example 35, where the first return signal includes a first linear component and a first nonlinear component and the second return signal includes a second linear component and a second nonlinear component. The generating of the combined signal is based on a linear combination of the first return signal and the second return signal.

Example 45. The user interface of example 44, where the first linear component is substantially opposite to the second linear component.

Example 46. The user interface of example 44, wherein the first nonlinear component and the second nonlinear component are based on nonlinear reflections from a contrast agent within the ROI.

Example 47. The user interface of example 37, wherein the first pulse and the second pulse are full-amplitude pulses.

Example 48: The user interface example 35, wherein the generating of the combined signal is the result of a summing of the first return signal and the second return signal.

Example 49. The user interface of example 35, where the transmitting the first ultrasound signal and the second ultrasound signal at the ROI includes determining a coverage area of the transmission of the first ultrasound signal and the second ultrasound signal and a bisector of the coverage area, where the first ultrasound signal and the second ultrasound signal are configured to converge at a point on the bisector.

Example 50. The user interface of example 35, wherein the user interface is further configured to cause the ultrasound scanner to generate a third ultrasound signal with the first polarity and a fourth ultrasound signal with the second polarity. The user interface is further configured to cause the ultrasound scanner to transmit the third ultrasound signal and the fourth ultrasound signal at the ROI and cause the ultrasound scanner to receive a third return signal based on the third ultrasound signal reflecting from the ROI and a fourth return signal based on the fourth ultrasound signal reflecting from the ROI. the user interface is further configured to cause the one or more processors to generate a second combined signal based on the third return signal and the second return signal and a second output based on the second combined signal.

Example 51. The user interface of example 50, where the user interface further causes the ultrasound scanner to determine a first coverage area of the transmission of the first ultrasound signal and the second ultrasound signal and a second coverage area of the transmission of the third ultrasound signal and the fourth ultrasound signal, the second coverage area overlapping the first coverage area at least in part.

### CONCLUSION

Embodiments of nonlinear contrast imaging with ultrasound as described herein are advantageous, as they provide for one or more of an increased SNR, contrast-to-noise ratio, contrast-to-tissue ratio, frame rate, and penetration in ultrasound scans with contrast and a minimization of unwanted imaging of surrounding tissue by attenuating the linear response from the ultrasound echoes. The techniques of nonlinear contrast imaging with ultrasound disclosed herein also increase frame rate by employing a multi-line rolling buffer method, such as by buffering multiple pulse outputs and combining the pulse output response signals where they overlap in the anatomy being scanned. The nonlinear contrast imaging with ultrasound provides increased scanning efficiency, improved patient experience, higher-fidelity scanning outcomes, and similar benefits.

While the present subject matter has been described in detail with respect to various specific example implementations thereof, each example is provided by way of explanation, not limitation of the disclosure. Those skilled in the art, upon attaining an understanding of the foregoing, can readily produce alterations to, variations of, and equivalents to such implementations. Accordingly, the subject disclosure does not preclude inclusion of such modifications, variations, and/or additions to the present subject matter as would be readily apparent to one of ordinary skill in the art. For instance, features illustrated or described as part of one implementation can be used with another implementation to yield a still further implementation. Thus, it is intended that the present disclosure cover such alterations, variations, and equivalents.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

While various embodiments of the disclosure are described in the foregoing description and shown in the drawings, it is to be distinctly understood that this disclosure is not limited thereto but may be variously embodied to practice within the scope of the following claims. From the foregoing description, it will be apparent that various changes may be made without departing from the spirit and scope of the disclosure as defined by the following claims.

What is claimed is:

1. An ultrasound device comprising:
an ultrasound scanner, the ultrasound scanner configured to:
generate a first ultrasound signal with a first polarity;
generate a second ultrasound signal with a second polarity, the second polarity being an inverse of the first polarity;
transmit the first ultrasound signal and the second ultrasound signal at a region of interest (ROI) of a subject;
receive a first return signal based on the first ultrasound signal reflecting from the ROI; and
receive a second return signal based on the second ultrasound signal reflecting from the ROI;
one or more processors; and
a memory, the memory storing instructions that, when executed by the one or more processors, cause the one or more processors to generate:
a combined signal based on the first return signal and the second return signal; and
an output based on the combined signal.

2. The ultrasound device of claim 1, wherein transmission of the first ultrasound signal and the second ultrasound signal occur at a same time.

3. The ultrasound device of claim 1, wherein:
the first ultrasound signal comprises a first pulse; and
the second ultrasound signal comprises a second pulse.

4. The ultrasound device of claim 1, wherein:
the first return signal comprises a first linear component and a first nonlinear component;
the second return signal comprises a second linear component and a second nonlinear component; and
the generating of the combined signal is based on a linear combination of the first return signal and the second return signal.

5. The ultrasound device of claim 4, wherein the first nonlinear component and the second nonlinear component are based on nonlinear reflections from a contrast agent within the ROI.

6. The ultrasound device of claim 1, wherein:
the configuring of the ultrasound scanner to transmit the first ultrasound signal and the second ultrasound signal at the ROI comprises determining:
a coverage area of transmission of the first ultrasound signal and the second ultrasound signal; and
a bisector of the coverage area; and
the first ultrasound signal and the second ultrasound signal are configured to converge at a point on the bisector.

7. The ultrasound device of claim 1, wherein:
the ultrasound scanner is further configured to:
generate a third ultrasound signal with the first polarity;
generate a fourth ultrasound signal with the second polarity;
transmit the third ultrasound signal and the fourth ultrasound signal at the ROI;
receive a third return signal based on the third ultrasound signal reflecting from the ROI; and
receive a fourth return signal based on the fourth ultrasound signal reflecting from the ROI; and
the instructions further cause the one or more processors to generate:
a second combined signal based on the third return signal and the second return signal; and
a second output based on the second combined signal.

8. The ultrasound device of claim 7, wherein the ultrasound scanner is further configured to determine:
a first coverage area of transmission of the first ultrasound signal and the second ultrasound signal; and
a second coverage area of transmission of the third ultrasound signal and the fourth ultrasound signal, the second coverage area overlapping the first coverage area at least in part.

9. A method for nonlinear contrast imaging with ultrasound, the method comprising:
generating, by an ultrasound scanner:
a first ultrasound signal with a first polarity; and
a second ultrasound signal with a second polarity, the second polarity being an inverse of the first polarity;
transmitting, by the ultrasound scanner, the first ultrasound signal and the second ultrasound signal at a region of interest (ROI) of a subject;
receiving, by the ultrasound scanner:
a first return signal based on the first ultrasound signal reflecting from the ROI; and
a second return signal based on the second ultrasound signal reflecting from the ROI; and
generating, by one or more processors:
a combined signal based on the first return signal and the second return signal; and
an output based on the combined signal.

10. The method of claim 9, wherein:
the first ultrasound signal comprises a first pulse; and
the second ultrasound signal comprises a second pulse.

11. The method of claim 10, wherein the generation of the combined signal represents a beamforming of the first pulse and the second pulse at the ROI.

12. The method of claim 10, further comprising changing, by the one or more processors, one or more parameters of the first ultrasound signal or the second ultrasound signal.

13. The method of claim 12, wherein the one or more parameters include a phase of the first pulse or the second pulse, an amplitude of the first pulse or the second pulse, a frequency of the first pulse or the second pulse, or a waveform of the first pulse or the second pulse.

14. The method of claim 12, wherein the changing of the one or more parameters is based on a machine-learned model.

15. The method of claim 9, further comprising:
generating, by the ultrasound scanner:
a third ultrasound signal with the first polarity; and
a fourth ultrasound signal with the second polarity;
transmitting, by the ultrasound scanner, the third ultrasound signal and the fourth ultrasound signal at the ROI;
receiving, by the ultrasound scanner:
a third return signal based on the third ultrasound signal reflecting from the ROI; and
a fourth return signal based on the fourth ultrasound signal reflecting from the ROI; and
generating, by the one or more processors:
a second combined signal based on the third return signal and the second return signal; and
a second output based on the second combined signal.

16. A user interface for an ultrasound device, the user interface configured to:
cause an ultrasound scanner of the ultrasound device to:
generate a first ultrasound signal with a first polarity;
generate a second ultrasound signal with a second polarity, the second polarity being an inverse of the first polarity;
transmit the first ultrasound signal and the second ultrasound signal at a region of interest (ROI) of a subject;
receive a first return signal based on the first ultrasound signal reflecting from the ROI; and
receive a second return signal based on the second ultrasound signal reflecting from the ROI; and
cause one or more processors of the ultrasound device to generate:
a combined signal based on the first return signal and the second return signal; and
an output based on the combined signal.

17. The user interface of claim 16, wherein:
the first ultrasound signal comprises a first pulse; and
the second ultrasound signal comprises a second pulse.

18. The user interface of claim 17, wherein the user interface is further configured to cause the one or more processors to change one or more parameters of the first ultrasound signal or the second ultrasound signal.

19. The user interface of claim 18, wherein the changing of the one or more parameters is based on at least one of the first return signal or the second return signal.

20. The user interface of claim 16, wherein the user interface is further configured to cause:
the ultrasound scanner to generate:
a third ultrasound signal with the first polarity; and
a fourth ultrasound signal with the second polarity;
the ultrasound scanner to transmit the third ultrasound signal and the fourth ultrasound signal at the ROI;
the ultrasound scanner to receive:
a third return signal based on the third ultrasound signal reflecting from the ROI; and
a fourth return signal based on the fourth ultrasound signal reflecting from the ROI; and
the one or more processors to generate:
a second combined signal based on the third return signal and the second return signal; and
a second output based on the second combined signal.
